(19) United States
(12) Patent Application Publication
(10) Pub. No.: US 2025/0134657 A1
(43) Pub. Date: May 1, 2025

(54) SYSTEMS AND DEVICES OF VALVULAR PROSTHETICS

(71) Applicant: EDWARDS LIFESCIENCES CORPORATION, Irvine, CA (US)

(72) Inventors: Christopher James Garete, Irvine, CA (US); Rani Abdullah Mahmoudi, Huntington Beach, CA (US); Matthew A. Peterson, Costa Mesa, CA (US); Wei Gan, Irvine, CA (US); Doo Wan Kim, Irvine, CA (US)

(21) Appl. No.: 19/009,850

(22) Filed: Jan. 3, 2025

Related U.S. Application Data
(63) Continuation of application No. PCT/US2023/026658, filed on Jun. 30, 2023.
(60) Provisional application No. 63/358,774, filed on Jul. 6, 2022.

(51) Int. Cl. A61F 2/24

(52) U.S. Cl. CPC A61F 2/2418; A61F 2/2427; A61F 2210/0004; A61F 2220/0008; A61F 2220/0025

(57) ABSTRACT

Systems, devices and methods of valvular prosthetics having one or more bioresorbable components are described. The bioresorbable components can resorb overtime, allowing the ingrowth of tissue in and around the valvular prosthetic.

### CROSS REFERENCE TO RELATED APPLICATIONS

This application is a continuation of PCT Application No. PCT/US2023/026658, entitled “Systems and Devices of Valvular Prosthetics,” filed Jun. 30, 2023, which claims benefit of U.S. Provisional Patent Application No. 63/358,774, entitled “Systems and Devices of Valvular Prosthetics,” filed Jul. 6, 2022, the disclosures of which are hereby incorporated by reference in their entirety for all purposes.

### FIELD OF TECHNOLOGY

The application is generally directed to devices and systems of valvular prosthetics, and more specifically to valvular devices and systems that provide bioresorbable components or improvements to blood flow.

### BACKGROUND

Prosthetic devices can be utilized to treat a variety of cardiac and circulatory disorders. For instance, prosthetic heart valves can be utilized to treat valvular disorders such as valvular insufficiency or aortic stenosis. A transcatheter technique may be used for introducing and implanting a prosthetic device in a manner that is less invasive and can reduce complications as compared with surgical procedures (e.g., open heart surgery). In a transcatheter technique, a prosthetic device can be mounted in a crimped state on the distal end portion of a delivery catheter and advanced through the vasculature of the patient until the prosthetic device reaches the implantation site. The prosthetic device at the catheter tip can then be expanded to its functional size at the treatment site, such as by inflating a balloon or utilizing self-expanding stent or frame. A prosthetic device can have a balloon-expandable, self-expanding, mechanically expandable frame, and/or a frame expandable in multiple or a combination of ways. Prosthetic devices used in this manner include transcatheter heart valves (THV's).

Existing prosthetic heart valves provide effective treatments; however, there may be complications due to undesirable forces resulting from the implantation of a prosthetic heart valve within a native heart. There may also be complications relating to uneven crimping, which can cause damage to the frame and/or difficulties during implantation. Prosthetic heart valves may also create the possibility of thrombus formation. Accordingly, there is a need for further improvements and refinements to the existing technology.

### SUMMARY OF THE DISCLOSURE

Systems and devices can be implanted within the vasculature and provide various benefits, including (but not limited to) resorption, improved crimping, improved blood flow, and improved manufacturing.

In some implementations, a frame is for use in a valvular prosthetic. The frame comprises a plurality of interconnected struts that form a tubular frame. The frame further comprises a plurality of bioresorbable elements. The bioresorbable elements may be located within a subset of the struts that form the frame.

Bioresorbable elements provide the ability to vary the size or shape of the frame after implantation. For example, it may be advantageous to provide a frame that has a fixed size upon initial implantation, but changes over time to a different size. By adjusting over time, it may be possible to reduce or eliminate forces on the heart or other surrounding tissue. Portions of the frame may become unnecessary over time as tissue ingrowth leads to firm fixation and thereby reduces the need for anchoring mechanisms on the frame.

For example, a valvular prosthetic may comprise a plurality of interconnected struts to form a tubular frame. The valvular prosthetic may further comprise an anchoring system attached to the tubular frame. The anchoring system may take the form of anchoring arms that anchor to surrounding tissue, such as be capturing native leaflets. Over time, the anchoring arms may become unnecessary. As such, the anchoring arms may be constructed to be bioresorbable. Alternatively, the anchoring arms may have tips that are bioresorbable.

In other implementations, a valvular prosthetic comprises a plurality of interconnected struts that form a tubular frame. The valvular prosthetic further comprises a skirt that is attached to the tubular frame. The skirt is bioresorbable. The skirt may be mounted on the inside of the frame or the outside of the frame. The skirt may also be mounted to both the inside and outside of the frame.

In some implementations, a valvular prosthetic comprises a plurality of interconnected struts that form a tubular frame. The valvular prosthetic further comprises a bioresorbable band that encircles the tubular frame. The band may be constructed to control frame expansion in a delayed release manner.

In some implementations, a valvular prosthetic comprises a plurality of interconnected struts that form a tubular frame. The valvular prosthetic further comprises an anchoring system attached to the tubular frame. The valvular prosthetic further comprises one or more bioresorbable barbs attached to the tubular frame or attached to the anchoring system. The barbs may provide a temporary anchoring mechanism, which resorbs over time.

In some implementations, the plurality of interconnected struts comprises shape-memory material that provides a radial force.

In some implementations, the frame further comprises a plurality of bioresorbable elements within a subset of the struts that form the tubular frame.

In some implementations, at least a subset of the plurality of bioresorbable elements are located at an interconnection point of two or more struts.

In some implementations, resorption of the bioresorbable elements results in weakening the radial force of the tubular frame.

In some implementations, the tubular frame further comprises a plurality of appendages that extend away from the tubular frame. Each appendage of the plurality of appendages is formed by a subset of the plurality of interconnected struts.

In some implementations, at least a subset of the plurality of bioresorbable elements are located within one or more appendages of the plurality of appendages.

In some implementations, the tubular frame is compressible for placement within a catheter of a transcatheter delivery system.

In some implementations, the frame further comprises a set of leaflets within the tubular frame. The set of leaflets are attached to the tubular frame or attached to the inner skirt. In some implementations, the frame is sterilized and packaged.

In some implementations, a valvular prosthetic comprises a plurality of interconnected struts to form a tubular frame that is self-expanding. The tubular frame comprises a shape-memory material. The valvular prosthetic comprises a constricting bioresorbable band that encircles the self-expanding tubular frame.

In some implementations, the valvular prosthetic is within a catheter of a transcatheter delivery system.

In some implementations, a valvular prosthetic comprises a set of columnar segments. Each columnar segment comprises a plurality of interconnected struts. The valvular prosthetic comprises a set of bioresorbable connectors. The set of bioresorbable connectors connect the set of columnar segments to form a tubular frame. Each columnar segment is in connection with two adjacent columnar sections via one or more bioresorbable connectors of the set.

In some implementations, a frame is for use in a valvular prosthetic. The frame comprises a plurality of interconnected struts that form a tubular frame having a plurality of cells. At least a subset of the plurality of cells is asymmetrical. The asymmetry of each cell of the subset of the plurality of cells that is asymmetrical may be formed by at least one cross strut that is asymmetrical.

In some implementations, the asymmetry of the at least one cross strut is formed by two curved portions that meet in a central apex. A first curved portion of the two curved portions has a greater radius and length than a second curved portion of the two curved portions.

In some implementations, a frame is for use in a valvular prosthetic. The frame comprises a plurality of interconnected struts the form a tubular frame. The tubular frame has an inflow end and outflow end. The frame further comprises a plurality of bioresorbable portions within a subset of the struts that form the tubular frame. The bioresorbable portions are at or near the outflow side.

In some implementations, resorption of the bioresorbable elements are at or near the outflow side results in a segmented outflow end with an ability to flex.

In some implementations, the frame is configured such that when the frame experiences a liquid flow and pressure through the frame, the segmented outflow end is capable of flexing outward to convert the outflow end from a slight-line shape to a flared-out shape.

In some implementations, a valvular prosthetic is provided for improving stagnated blood flow. The valvular prosthetic comprises a plurality of interconnected struts that form a tubular frame. The tubular frame has an inflow side and outflow side. The valvular prosthetic further comprises an inner luminal wall attached to the tubular frame. The valvular prosthetic further comprises a set of leaflets within the tubular frame. The set of leaflets is attached to the tubular frame or attached to the inner luminal wall and separate the inflow side from the outflow side of the tubular frame. The valvular prosthetic further comprises a set of one or more inflatable bags attached to the inner luminal wall on the outflow side of the frame.

In some implementations, each bag of the set of one or more inflatable bags is composed of a biocompatible flexible material.

In some implementations, each bag of the set of one or more inflatable bags is filled with a compressed fluid component that changes volume based on pressure.

In some implementations, the set of one or more inflatable of bags are configured such that when the valvular prosthetic experiences a liquid flow and pressure through the inner luminal wall, each bag of the set of one or more inflatable bags is capable of expanding into an inflated state.

In some implementations, the valvular prosthetic is implanted within vasculature of an animal and wherein the liquid flow and pressure is systolic blood flow and pressure.

In some implementations, a valvular prosthetic is provided for improving stagnated blood flow. The valvular prosthetic comprises a plurality of interconnected struts that form a tubular frame. The tubular frame has an inflow side and outflow side. The valvular prosthetic further comprises an inner luminal wall attached to the tubular frame. The valvular prosthetic further comprises a set of leaflets within the tubular frame. The set of leaflets is attached to the tubular frame or attached to the inner luminal wall and separate the inflow side from the outflow side of the tubular frame. The valvular prosthetic further comprises a set of one or more free-flowing sheets attached to the inner luminal wall on the outflow side of the frame. Each free-flowing sheet of the set of one or more free-flowing sheets has at least one free edge.

In some implementations, each free-flowing sheet of the set of one or more free-flowing sheets is composed of a biocompatible flexible material.

In some implementations, the set of one or more free flowing sheets are configured such that when the valvular prosthetic experiences a liquid flow and pressure through the inner luminal wall, each free-flowing sheet of the set of one or more free-flowing sheets is capable of moving with the liquid flow.

In some implementations, a valvular prosthetic is for improving stagnated blood flow. The valvular prosthetic comprises a plurality of interconnected struts that form a tubular frame. The tubular frame has an inflow side and outflow side. The valvular prosthetic further comprises an inner luminal wall attached to the tubular frame. The valvular prosthetic further comprises a set of leaflets within the tubular frame. The set of leaflets is attached to the tubular frame or attached to the inner luminal wall and separate the inflow side from the outflow side of the tubular frame. The valvular prosthetic further comprises a plurality of flexible magnetically driven microprotrusions that are linearly aligned in the direction of flow, and attached to the inner luminal wall. Each flexible magnetically driven microprotrusion of the plurality of flexible magnetically driven microprotrusions has a positive magnetic pole and negative magnetic pole at a tip of the microprotrusion.

In some implementations, the plurality of flexible magnetically driven microprotrusions comprises a larger driver microprotrusion.

In some implementations, each microprotrusion tip of the plurality of flexible magnetically driven microprotrusions has a particular pole alignment such that each the pole face of each tip is the same charge as the adjacent tip pole face.

In some implementations, the larger driver microprotrusion can stimulate flexing of the rest of the plurality of flexible magnetically driven microprotrusions.

In some implementations, a frame system is for use within a valvular prosthetic. The frame system comprises a plurality of interconnected struts that form a tubular frame that is self-expanding. The frame system comprises a plurality of anti-torsion elements. Each anti-torsion element is a protrusion that extends laterally from at least a subset of the plurality of interconnected struts.

In some implementations, each anti-torsion element is attached to a strut that is in an area of the frame that has a low density of struts.

In some implementations, each anti-torsion element is fabricated as part of the frame design.

In some implementations, each anti-torsion element is attached and secured to a strut by a means of attachment.

In some implementations, at least a subset of anti-torsion elements of the plurality abuts or nearly abuts an adjacent strut when the frame is crimped.

In some implementations, each anti-torsion element laterally extends a length between about 1.1× and 5× the lateral width of the strut.

In some implementations, each anti-torsion element has a vertical length between about 2% to 20% of the length of the strut.

In some implementations, at least a subset of the plurality of anti-torsion elements comprises a marker for visualization.

In some implementations, the frame system further comprises a catheter. The catheter houses the tubular frame. Each anti-torsion element mitigates the ability of a strut from twisting or contorting during loading of the tubular frame into the catheter.

In some implementations, the catheter comprises an inner face having a polygonal contour.

In some implementations, the tubular frame comprises a number of columnar segments, and wherein the number of columnar segments is equal to the number of sides of the polygonal contour.

In some implementations, at least a subset of the plurality of interconnecting struts abuts or nearly abuts a side of the polygonal contour.

In some implementations, a method is for releasing a frame via a transcatheter technique. The method comprises delivering a catheter and a tubular frame that is self-expanding to a site where the tubular frame is to be installed. The tubular frame is loaded within the catheter. The tubular frame comprises a shape-memory material for providing a radial force, a plurality of interconnected struts, and a plurality of anti-torsion elements. Each anti-torsion element is a protrusion that extends laterally from at least a subset of the plurality of interconnected struts. The method comprises distally advancing the tubular frame out of the catheter, resulting in an expansion of the tubular frame. The method further comprises proximally reloading the tubular frame into the catheter, resulting in a crimping of the tubular frame.

In some implementations, each anti-torsion element is attached to a strut that is in an area of the frame that has a low density of struts.

In some implementations, each anti-torsion element laterally extends a length between about 1.1 to 5 times the lateral width of the strut.

In some implementations, at least a subset of the plurality of anti-torsion elements comprises a marker for visualization. The method further comprises visualizing distally advancing or the proximally reloading of the self-expanding tubular frame via the marker for visualization and a visualization technique.

In some implementations, a frame system is for use within a valvular prosthetic. The frame system comprises a plurality of interconnected struts that form a tubular frame that is self-expanding. The tubular frame comprises a shape-memory material for providing a radial force. The frame system comprises a catheter. The catheter houses the tubular frame. The catheter comprises an inner face having a polygonal contour.

In some implementations, the tubular frame comprises a number of columnar segments. The number of columnar segments is equal to the number of sides of the polygonal contour.

In some implementations, at least a subset of the plurality of interconnecting struts abuts or nearly abuts a side of the polygonal contour.

In some implementations, the frame system further comprises a plurality of anti-torsion elements. Each anti-torsion element is a protrusion that extends laterally from at least a subset of the plurality of interconnected struts.

In some implementations, each anti-torsion element is attached to a strut that is in an area of the frame that has a low density of struts.

In some implementations, at least a subset of anti-torsion elements of the plurality abuts or nearly abuts an adjacent strut when the frame is crimped.

In some implementations, wherein each anti-torsion element laterally extends a length between about 1.1 to 5 times the lateral width of the strut.

In some implementations, wherein each anti-torsion element has a vertical length between about 2% to 20% of the length of the strut.

In some implementations, a method is for releasing a frame via a transcatheter technique. The method comprises delivering a catheter and a tubular frame that is self-expanding to a site where the tubular frame is to be installed. The tubular frame is loaded within the catheter. The catheter comprises an inner face having a polygonal contour. The tubular frame comprises a shape-memory material for providing a radial force and comprises a plurality of interconnected struts. The method comprises distally advancing the tubular frame out of the catheter, resulting in expansion of the tubular frame. The method comprises proximally reloading the tubular frame into the catheter, resulting in crimping of the tubular frame.

In some implementations, the frame comprises a number of columnar segments, and wherein the number of columnar segments is equal to the number of sides of the polygonal contour.

In some implementations, at least a subset of the plurality of interconnecting struts abuts or nearly abuts a side of the polygonal contour.

In some implementations, the tubular frame further comprises a plurality of anti-torsion elements. Each anti-torsion element is a protrusion that extends laterally from at least a subset of the plurality of interconnected struts.

In some implementations, a cover is for the fabrication of a tubular frame having a plurality of circumferences along the proximal-distal axis of the frame. The cover comprises a precut sheet comprising a plurality of columnar segments that form a flower shape. Each columnar segment has a first end, a second end, and two lateral edges. Each columnar segment is laterally connected to two adjacent segments at the first end. Each columnar segment is not laterally connected from the two adjacent segments at the second end and for a majority of each of the two lateral edges. In some implementations, the precut sheet is composed of fabric, tissue, or film.

In some implementations, a system comprises a tubular frame that extends a long a proximal-distal axis. The tubular frame comprises a plurality of columnar segments, each segment extending along the proximal-distal axis. The tubular frame comprises a plurality of circumference lengths along the proximal distal axis. The tubular frame comprises a proximal end and a distal end. The system comprises a cover surrounding the tubular frame. The cover is composed of a precut sheet in a flower shape. The cover comprises a plurality of columnar segments. Each columnar segment has a first end, a second end, and two lateral edges. The two lateral edges of each columnar segment is laterally connected to lateral edges of two adjacent segments at the first end by the precut flower shape. The two lateral edges of each columnar segment is laterally connected to lateral edges of two adjacent segments at the second end by a means of attachment.

In some implementations, the majority of each of the two lateral edges of each columnar segment is laterally connected to lateral edges of two adjacent segments by the means of attachment. In some implementations, the means of attachment comprises one of: stitching, staples, or an adhesive.

In some implementations, the first end of the cover surrounds the proximal end of the tubular frame and the second end of the cover surrounds the distal end of the tubular frame.

In some implementations, the first end of the cover surrounds the distal end of the tubular frame and the second end of the cover surrounds the proximal end of the tubular frame.

In some implementations, the lateral edges of each columnar segment of the cover contours to match the plurality of circumferences along the proximal-distal axis of the frame.

In some implementations, the number of columnar segments of the plurality of segments of the cover match the number of columnar segments of the frame.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 provides an illustration of the human heart.

FIG. 2A and FIG. 2B provide an example of a tubular frame having bioresorbable portions for use in a valvular prosthetic.

FIG. 3A provides an example of a bioresorbable anchoring system for use in a valvular prosthetic.

FIGS. 3B and 3C provide an exemplary schematic of a bioresorbable anchoring system in the context of a local site of implantation.

FIG. 4A provides an example of a set of bioresorbable barbs for use in a valvular prosthetic.

FIGS. 4B and 4C provide an exemplary schematic of a set of bioresorbable barbs in the context of a local site of implantation.

FIG. 5 provides examples of a bioresorbable outer skirt and bioresorbable inner skirt for use in a valvular prosthetic.

FIG. 6 provides an example of a fluffy bioresorbable outer band for use in a valvular prosthetic.

FIG. 7A provide an example of a bioresorbable band for controlling frame expansion for use in a valvular prosthetic.

FIG. 7B provides an exemplary schematic of expansion of a frame after a controlling bioresorbable band upon resorption.

FIG. 8A provides an example of a set of bioresorbable connectors for linking multiple frame segments for use in a valvular prosthetic.

FIG. 8B provides an exemplary schematic of a tubular frame having multiple segments that were linked by a set of bioresorbable connectors after the set of bioresorbable connectors is resorbed.

FIG. 9 provides an example of a frame with asymmetrical strut design for use in a valvular prosthetic.

FIG. 10A provides an example of a frame with bioresorbable portions near the outflow portion for use in a valvular prosthetic.

FIGS. 10B and 10C provide an exemplary schematic of a valvular prosthetic having a frame with resorbed bioresorbable portions near the outflow portion during systole and diastole.

FIGS. 11A and 11B provide an exemplary schematic of a valvular prosthetic having a set of one or more inflatable bags for improving stagnated blood flow.

FIG. 12 provide an exemplary schematic of a valvular prosthetic having a set of sheets with a free edge for improving stagnated blood flow.

FIGS. 13A and 13B provide an exemplary schematic of a valvular prosthetic having a set of magnetic microprotrusions for improving stagnated blood flow.

FIGS. 14A and 14B provide an example of a frame with a plurality of elements to prevent torsion.

FIGS. 15A and 15B provide an example of a frame within a catheter, focusing on a single columnar segment of the frame. FIG. 15B provides a cross-sectional view of the catheter and frame of FIG. 15A.

FIGS. 16A and 16B provide an example of a frame comprising anti-torsion elements within a catheter, focusing on a single columnar segment of the frame. FIG. 16B provides a cross-sectional view of the catheter and frame of FIG. 16A.

FIGS. 17A and 17B provide an example of a frame within a catheter comprising an inner face with a polygonal contour, focusing on a single columnar segment of the frame. FIG. 17B provides a cross-sectional view of the catheter and frame of FIG. 17A.

FIGS. 18A and 18B provide an example of a precut cover to facilitate attaching the cover to a frame.

### DETAILED DESCRIPTION

Turning now to the drawings, various valvular systems and devices to improve functionality, host interaction, blood flow, prosthetic delivery, and/or prosthetic manufacturing are described. The valvular systems and devices can be utilized as a prosthetic (or as part of a prosthetic delivery and securement system) for replacing the function of any of the four heart valves: aortic valve, mitral valve, tricuspid valve, or pulmonary valve. In some implementations, a valvular system or device has one or more bioresorbable components. In some implementations, a valvular system or device has one or more components for improving blood flow. In some implementations, a valvular system or device has one or more components for improving valve crimping into and/or release from a catheter. In some implementations, a valvular system or device has one or more components for improving attachment of a cover to a frame.

The described systems, devices and methods should not be construed as limiting in any way. Instead, the present disclosure is directed toward all novel and nonobvious features and aspects of the various disclosed components, alone and in various combinations and sub-combinations with one another. The disclosed systems, devices and methods are not limited to any specific aspect, feature, or combination thereof, nor do the disclosed systems, devices and methods require that any one or more specific advantages be present or problems be solved.

Various components of systems and devices and examples of prosthetic valves or transcatheter valves are disclosed herein, and any combination of these options can be made unless specifically excluded. For example, any of the bioresorbable components disclosed, can be used with any other type of the other bioresorbable components, even if a specific combination is not explicitly described. Likewise, the different constructions and features of components of devices, methods and systems, such as (for example) elements for reducing torsion and bioresorbable components, can be mixed and matched. As another example, any bioresorbable component type/feature, valve type/feature, tissue cover type/feature, catheter type/feature etc., can be combined even if not explicitly disclosed. In short, individual components of the disclosed systems and devices can be combined unless mutually exclusive or physically impossible.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed systems, devices and methods can be used in conjunction with other systems, devices and methods.

The terms “proximal” and “distal” as used throughout the description relate to a catheter system axis, in which the end where the procedure is performed is the distal end and the opposite end where the catheter system is controlled is the proximal end. Accordingly, the distal end of the catheter system is the leading end that first traverses into the body and first reaches the procedure site. Conversely, the proximal end of the catheter system is the portion that remains extracorporeal. Likewise, a distal movement along the catheter axis would be movement of a component in a direction towards a site of procedure and a proximal movement along the catheter axis would be movement of a component in an opposite direction. Although these terms have a relationship with a site of procedure, it is to be understood that these terms are used for reference and the site of procedure does not need to be present when interpreting the components or movements of the devices and systems described herein.

Various systems and devices for repair are utilized for the purpose of performing a procedure within a recipient. Recipients include (but are not limited to) patients, animal models, cadavers, or anthropomorphic phantoms. Accordingly, in addition to methods of treating patients, the systems and devices can be utilized in training or other practice procedures upon animal models, cadavers, or anthropomorphic phantoms. Further, the techniques, methods, operations, steps, etc. described or suggested herein can be performed on a living animal or on a non-living simulation, such as on a cadaver, cadaver heart, anthropomorphic ghost, simulator (e.g., with the body parts, tissue, etc. being simulated), etc.

The described systems and devices can be sterilized, which can be performed using gamma irradiation, gas plasma, aldehydes, ethylene oxide, and/or e-beam. The systems or devices can be further treated with a formaldehyde bioburden reduction process. After preparation, the systems and devices can be stored within a container, which can be hermetically sealed or otherwise kept sterile.

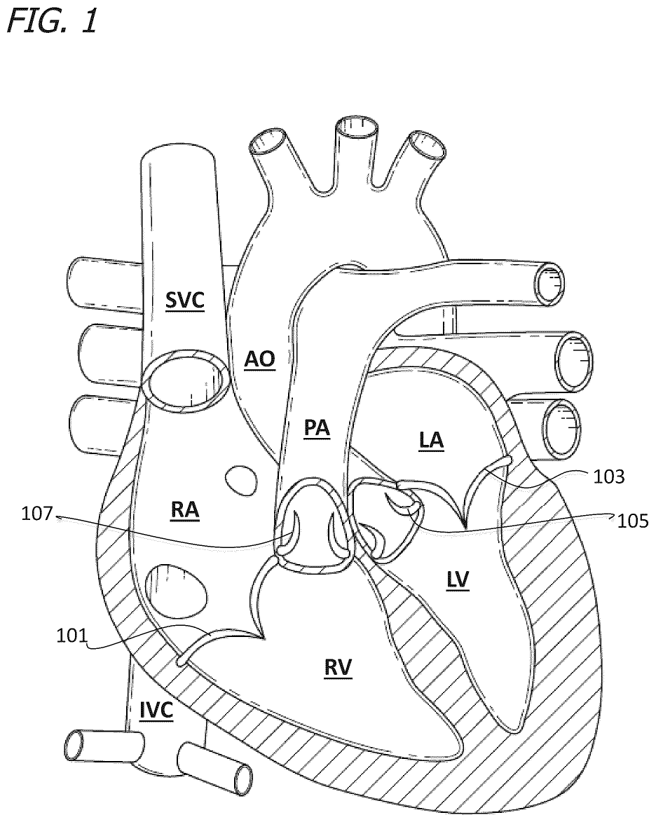

FIG. 1 is a cutaway view of the human heart in a systolic phase. The right ventricle (RV) and left ventricle (LV) are separated from the right atrium (RA) and left atrium (LA), respectively, by the tricuspid valve 101 and mitral valve 103; i.e., the atrioventricular valves. Additionally, the aortic valve 105 separates the LV from the ascending aorta (AO) and the pulmonary valve 107 separates the RV from the pulmonary artery (PA). Each of these valves has flexible leaflets extending inward across the respective orifices that come together or “coapt” in the flowstream to form the one-way, fluid-occluding surfaces.

The RA receives deoxygenated blood from the venous system through the SVC and the IVC, the former entering the RA from above, and the latter from below. During the diastolic phase, or diastole, the deoxygenated blood from the IVC, and SVC that has collected in the RA passes through the tricuspid valve 101 and into the RV as the RV expands. Likewise, oxygenated blood from pulmonary veins that has collected in the LA passes through the mitral valve 103 and into the LV as the LV expands. In the systolic phase, or systole, the RV contracts to force the deoxygenated blood collected in the RV through the pulmonary valve 107 into the pulmonary artery and lungs. Likewise, the LV contracts to force the deoxygenated blood collected in the LV through aortic valve 105 into the aorta and to the peripheral cardiovascular system.

The systems and device described within the present application are described, for illustration, may be utilized within for replacement or repair of any native valve or within the cardiac system. A native valve may need replacement or repair if, for example, the valve is stenotic and/or suffer from insufficiency and/or regurgitation. The systems and devices described herein can be used in various areas whether explicitly described herein or not, as treatment for a defective native valve or another cardiovascular disorder.

Various valvular prosthetic systems and devices can have one or more bioresorbable components, which can provide various improved functionalities, such as (for example) providing an ability to improve integration at a site of implantation. Typically, when a valvular prosthetic is implanted, several components such as (for example) frames, anchors, and skirts provide a benefit to enable prosthetic installation and/or valvular function immediately post implantation. Several of these components, however, are not required over time when a valve integrates with the local anatomy. Further, some of these components have potential to cause harm to the local anatomy. Accordingly, after implantation, one or more components can be bioresorbable such that the one or more components resorb over a period of time, resulting in a valve free of various components, such as (for example) frames, anchors, and skirts. Accordingly, the local tissue at the site of implantation can more freely grow in and around the valve, integrating the valve with the host's tissue.

The valvular prosthetic comprising a bioresorbable portion can be a replacement valve prosthetic for replacing any of the valves of the heart: tricuspid, pulmonary, mitral, or aortic. A replacement valve can further comprise one or more of: an inner skirt, an outer skirt, and a set of leaflets. The tubular frame can have an inlet end portion and outlet end portion with set of leaflets disposed therebetween and within the interior lumen of the prosthetic tubular frame for providing unidirectional blood flow through the valve. The set of leaflets can comprise 2, 3, 4, or more leaflets, which can be composed of pericardial tissue derived from bovine, porcine, or human donor.

A valvular prosthetic can be crimped and contained within a sheath of a transcatheter system for delivering the tubular frame. In some implementations, a prosthetic heart valve is crimped and contained within a sheath of a transcatheter system. A valvular prosthetic can be delivered to a site of installation by any appropriate approach, including (but not limited to) transfemoral, transjugular, subclavian, transapical, or transaortic approach. In some implementations, a valvular prosthetic is replacing the tricuspid valve and can be delivered via the femoral vein and inferior vena cava or via the jugular vein and the superior vena cava into the right atrium. In some implementations, a valvular prosthetic is replacing the mitral valve and can be delivered via the femoral vein, through the inferior vena cava into the right atrium, and traverse through the atrial septum into left atrium.

A bioresorbable component is to mean that the component is biodegradable over time, such that the component breaks down and degrades within body. Any biocompatible material can be utilized for the various components described herein. Examples of biocompatible and biodegradable material for use as a bioresorbable components include (but are not limited to) poly(glycolic acid) (PGA), poly(lactic acid) (PLA), poly(L-lactic acid) (PLLA), poly(D-lactic acid) (PDLA), poly(D,L-lactic acid) (PDLLA), poly(lactic-co-glycolic acid) (PGLA), poly(β-hydroxybutyrate-co-β-hydroxy valerate) (PHBV), poly(hydroxy butyrate) (PHB), polycaprolactone (PCL), polycyanoacrylates (e.g., poly(octyl cyanoacrylate) (POCA)), polyanhydrides (e.g, poly(fumaric-co-sebacic acid) (p(FASA)), and poly(propylene fumarate) (PPF). The composition of bioresorbable materials by means of various combinations and percentages can be controlled to yield desirable results. For example, degradation time can be controlled via the selection and composition of materials: PGA resorbs within one to two months, PLA/PGA (80/20) resorbs within one to two years, and PLLA resorbs in more than five years.

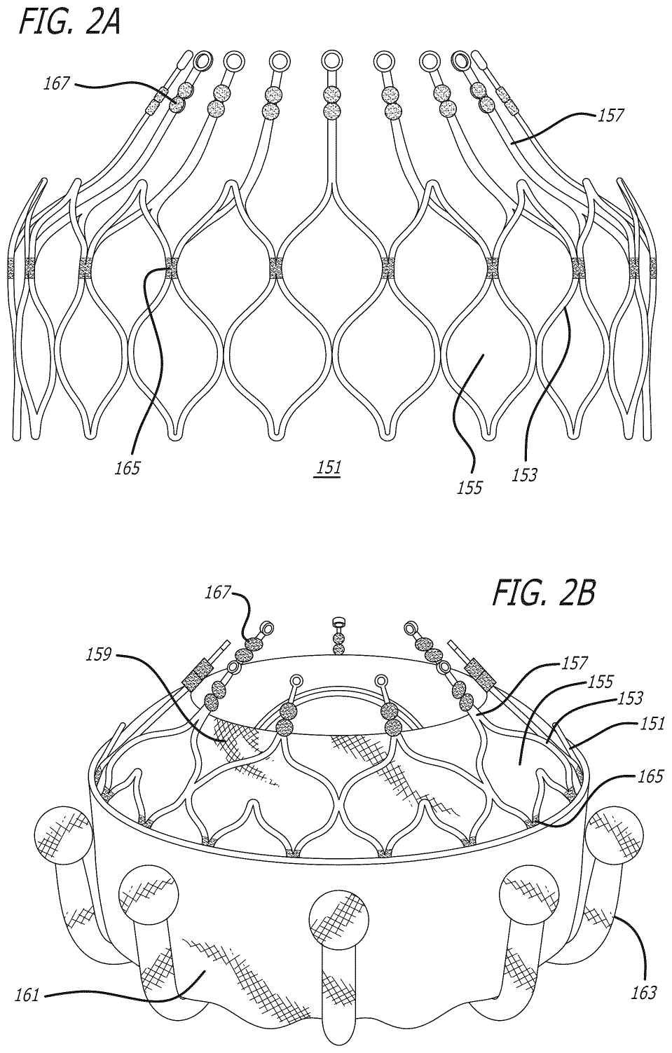

Provided in FIGS. 2A and 2B are non-exhaustive examples that illustrate bioresorbable portions of frames of a valvular prosthetic. The tubular frame 151 can be composed of a memory-shape material (e.g., nitinol) and comprise a plurality of struts 153 interconnected to form cells 155 that can form a frame base 157. Struts 153 can also extend from base 157 to form a number of appendages 157. It should be understood that any frame design is contemplated, and any interconnectivity of struts to form cells and/or appendages can be utilized. FIG. 2B further includes an inner skirt 159, an outer skirt 161, and several anchoring arms 163, which can house a set of leaflets on the inner skirt to form a valvular prosthetic for installation. Leaflets can be composed of any appropriate material, such as (for example) bovine pericardium, porcine pericardium, or human donor pericardium. When replacing a tricuspid valve or mitral valve, anchoring arms 163 can be utilized as an anchor for anchoring the replacement valve. Curves of each anchoring arm 163 can be utilized to latch onto chordae tendineae of the tricuspid valve or mitral valve, resisting atrial and ventricular migration and helping to hold the replacement valve in place when installed.

Frames that are composed of a memory-shape material, such as nitinol) can continually provide a radial force based on its shape memory. The radial force can push outwardly against the host's tissue at the site of installation. At the time of installation, the radial force of the frame in conjunction with a constrictive force of the local tissue helps ensure that the frame anchors and maintains a valvular prosthetic in its installed position. Over a period of time, however, the radial force is no longer needed as local tissue can grow into and round the valvular prosthetic to anchor and maintain the position. The continued radial force can cause discomfort, or cause injury, or drive undesirable anatomical remodeling, or negatively impact heart function as it consistently combats the constrictive force of the inner tissue and tissue ingrowth.

To counter the issue of continual radial force, a frame can comprise a number of struts 153 that have an element that is bioresorbable. For example, frame 151 comprises a number of struts 153 that have a bioresorbable elements 165 and 167 that can resorb over time. The bioresorbable element that is can be portion of the strut itself and/or a bioresorbable connector that connects one or more struts. As can be inferred from the depicted example, bioresorbable element 165 is located centrally on frame 151 (e.g., on a central connecting point among struts) and thus can decompose after installation, which can weaken the radially expanding frame and decrease the radial force it provides. Any method of expanding the frame can be utilized, including expanding via a balloon, mechanical expansion, or utilization of a shape-memory material (e.g., nitinol).

In a similar manner, bioresorbable element 167 is located peripherally on frame 151 (e.g., on a peripheral strut appendage) can decompose after installation to weaken the extended appendages, removing the most extended portions of the frame and reducing its overall size. It should be understood that various portions of the frame can be bioresorbable to weaken the radial force of a frame or an extended appendage and is not limited to the precise locations depicted in FIGS. 2A and 2B. In particular, any central portion of a strut or any connecting point between struts can be composed of bioresorbable material.

Frame 151 can also be composed entirely of bioresorbable materials. Accordingly, once the valvular prosthetic is installed at the site of implantation, a bioresorbable frame can resorb as tissue ingrowth occurs. The tissue ingrowth can provide the structure needed to support the valve function of the valvular implant such that the frame is no longer needed. In some implementations, a frame composed entirely of bioresorbable materials is detachable.

When installing a valvular prosthetic, anchors can be utilized to secure the prosthetic in its installed location. Over some period of time after installation, tissue ingrowth can hold and maintain the prosthetic in the installed location, and as such, anchors and barbs no longer needed provide a needed function. Further, anchors and barbs can cause disturbances to the local tissue as their bulkiness or sharp points can push into tissue walls or other local anatomies.

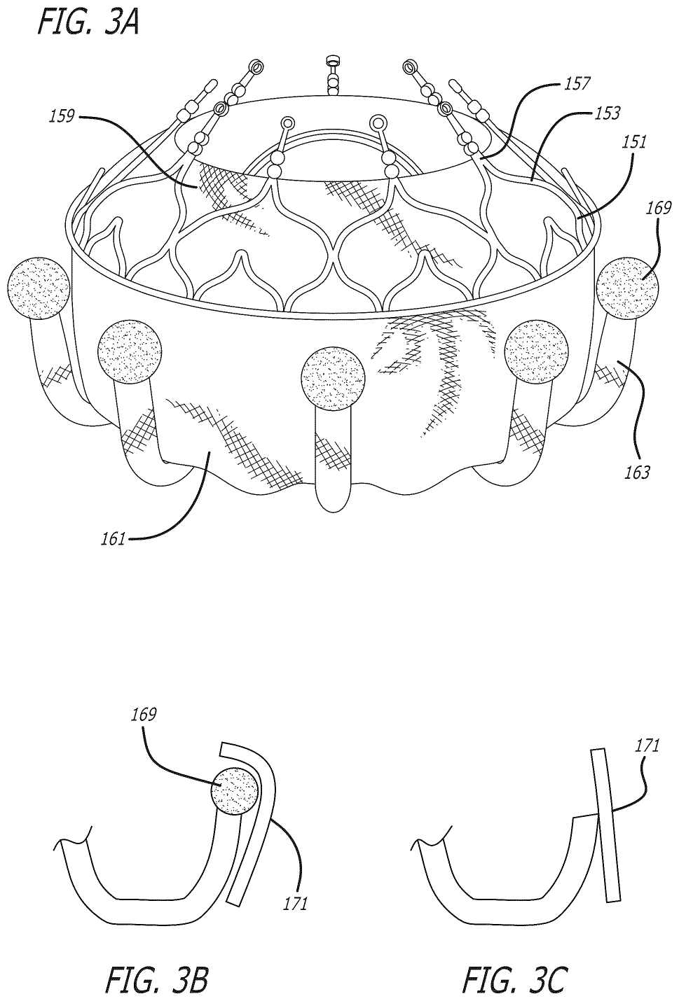

Provided in FIG. 3A is an example of a valvular prosthetic in which an anchoring system comprises bioresorbable portions. Similar to FIGS. 2A and 2B, frame 151 can be formed, at least in part, of memory-shape material (e.g., nitinol) and comprise a number of struts 153 interconnected to form a number of cells 155 that can form a frame base 157. Struts 153 can also extend from base 157 to form a number of appendages 157. The valvular prosthetic further includes an inner skirt 159, an outer skirt 161, and an anchoring system 163. The valvular prosthetic can further comprise a set of leaflets. Here, anchoring system 163 comprises a plurality of anchoring arms having bioresorbable anchor tips 169. Bioresorbable anchor tips 169 can help hold the native leaflets and maintain the valvular prosthetic in its installed location as tissue grows into the prosthetic.

FIGS. 3B and 3C provide an example of utility of bioresorbable anchor tips. For example, it has been discovered that when the valvular prosthetic is installed, anchor tip 169 can cause issues by protruding into local tissue 171 (FIG. 3B). To solve this problem, bioprosthetic anchor tip 169 can resorb over time such that it no longer protrudes into local tissue 171 (FIG. 3C) and thus preventing damage and/or remodeling of the local tissue.

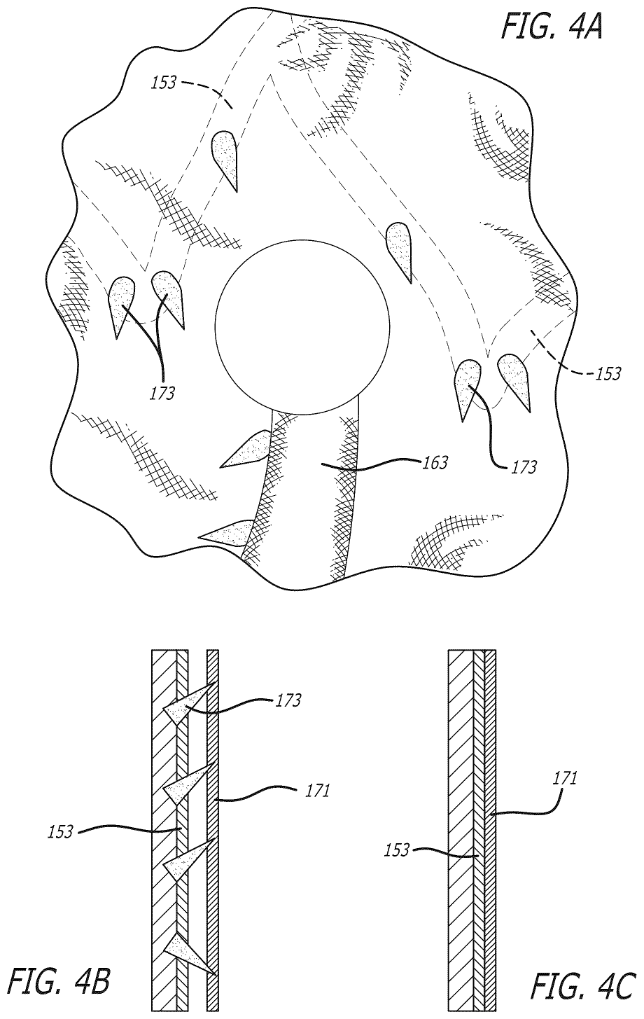

FIG. 4A provides a zoomed in view of a portion of a valvular prosthetic, showing an anchoring arm 163 with an anchor and a portion of frame 151 behind an outer skirt 161. Also shown is a number of bioresorbable barbs 173 extending from frame 151 and/or the anchoring arm 163. Bioresorbable barbs 173 can help secure the valve at the implantation site by engaging local tissue 171 (FIG. 4B). Much like the anchor, barbs are not needed when the tissue grows into the prosthetic and secures the valve via the tissue ingrowth. Over time the bioresorbable barbs 173 can resorb, leaving local tissue 171 free of barbs (FIG. 4C) and allowing for better healing and ingrowth.

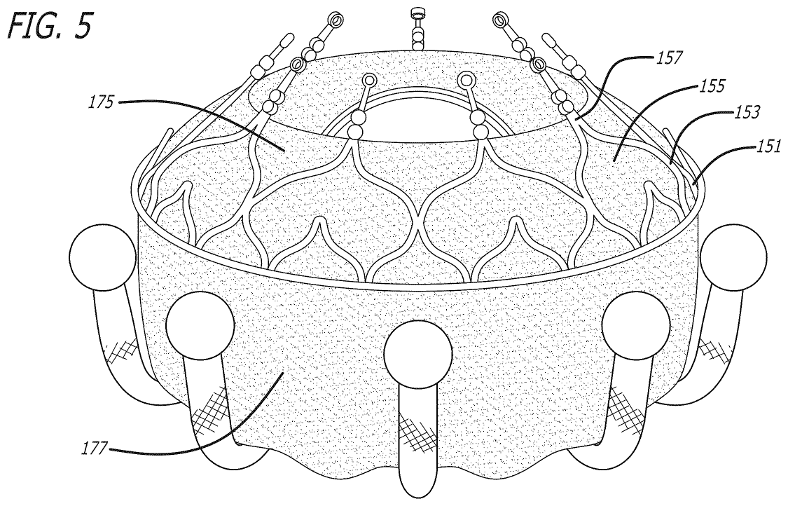

Generally, a skirt is utilized on a valvular prosthetic to help promote unidirectional flow and prevent paravalvular leakage. Provided in FIG. 5 is a valvular prosthetic with an inner skirt 175 and outer skirt 177 made of bioresorbable materials, which can be impermeable or semi-permeable to blood and constituents of the circulatory system. Bioresorbable inner skirt 175 can be impermeable such that blood can flow through the valve without any leakage. Bioresorbable outer skirt 177 can integrate with the local tissue to promote tissue regrowth and reendothelialization. After implantation, bioresorbable inner skirt 175 and bioresorbable outer skirt 177 can resorb as tissue grows into the valve to provide a sealing effect with the local tissue. Although FIG. 5 depicts both a valvular prosthetic with both a bioresorbable inner skirt and a bioresorbable outer skirt, various valvular prosthetics can be constructed as either just a bioresorbable inner skirt or just a bioresorbable outer skirt and thus should not be limited to valvular prosthetics having both bioresorbable inner and outer skirts.

To promote blood flow and prevent tissue ingrowth, the bioresorbable material of the inner face of the inner skirt can be fluorinated or otherwise made more hydrophobic.

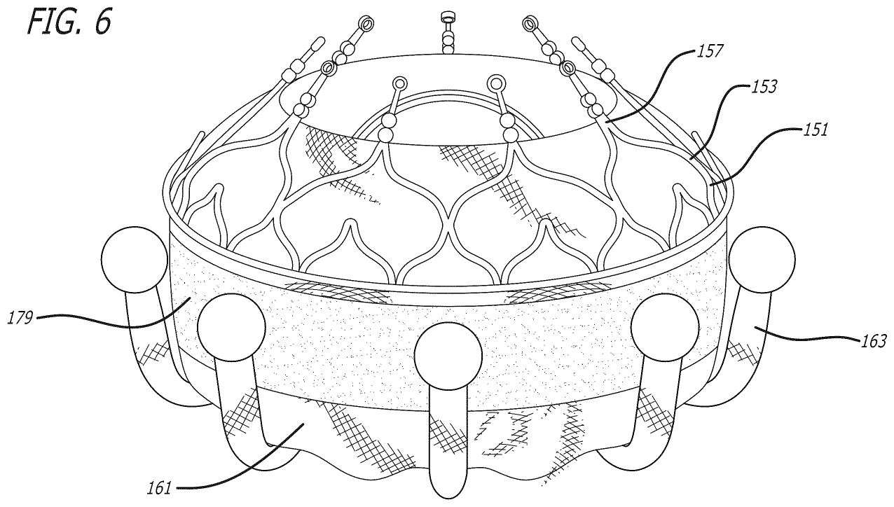

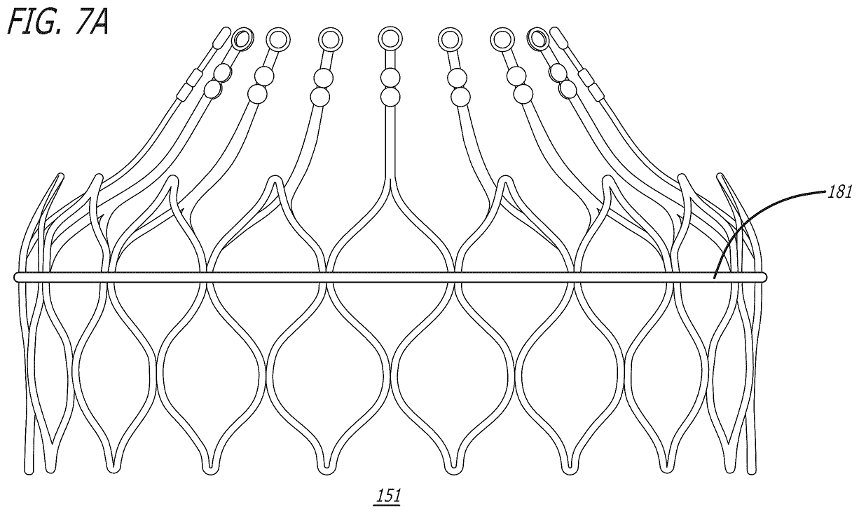

To promote tissue ingrowth, an outer valvular skirt can be composed of a fluffy material having a thickness and high porosity, which allows for blood integration and clotting, which can help formulate a seal to prevent paravalvular leakage and also promote tissue ingrowth. Accordingly, outer skirt 177 can be composed of a bioresorbable fluffy material. Alternatively, or in addition, an outer band 179 composed of a bioresorbable fluffy material can be utilized, which can be placed on top of an outer skirt 161 (FIG. 6). Fluffy bioresorbable outer band 179 can fully or partially encircle a valvular prosthetic for promoting blood integration and clotting. After implantation of the valvular prosthetic, fluffy bioresorbable outer skirt 177 or fluffy bioresorbable outer band 179 can resorb as tissue grows into the prosthetic and a seal is formed. In some implementations, the external face of the outer skirt and/or outer band has been coated with a reendothelialization-inducing biologic, such as (for example) amino acids (e.g., lysine or ornithine), saccharides (e.g., hyaluronic acid, fibronectin, chitosan), structural proteins (e.g., collagen, elastin), growth factors (e.g., VEGF), and combinations thereof.

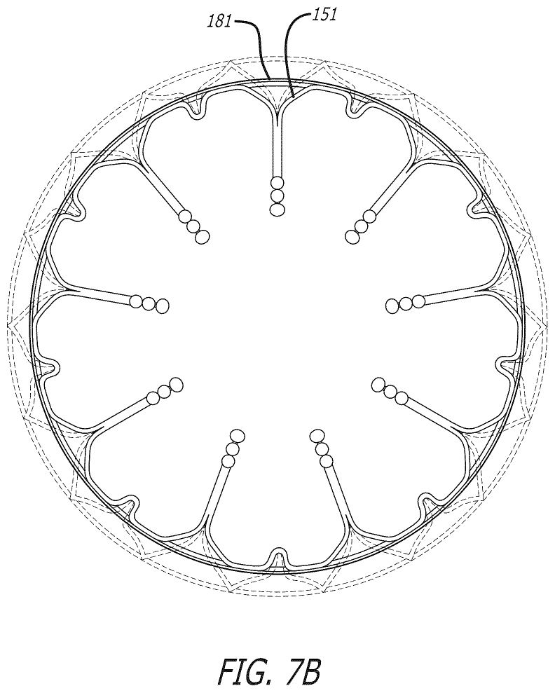

Bioresorbable materials can also be utilized to help control expansion and/or constriction of a valvular frame. Provided in FIG. 7A is a frame 151 that can be expanded and a bioresorbable band 181 that encircles the frame. Bioresorbable band 181 can prevent the expansion of frame 151 beyond its circumference, ensuring the frame is installed at a circumference as set by the bioresorbable band. After installation at the site of implantation, bioresorbable band 181 can resorb freeing the frame from constriction. In some implementations, frame 151 is composed of a shape-memory material (e.g., nitinol) and is self-expanding. Thus, when bioresorbable band 181 resorbs, the frame is free to expand outwardly (FIG. 7B).

In response to corrected valvular leak, the local anatomy of the patient anatomy has a propensity to return to normal dimensions (commonly referred to positive remodeling). The remodeling process shrinks the atrium, the annulus, and the ventricle, thereby providing relief to the heart and restoring more sustainable, physiologic function. Because many valvular prosthetic frames are self-expanding, a radial force due to the expansion mitigates this natural positive remodeling. It would thus be ideal to provide a frame that provides radial expansion during installation that later stops providing radial forces to allow positive remodeling. In some implementations, a valvular prosthetic frame can comprise a plurality of segments along the outer diameter. Each of the segments can be connected via bioresorbable materials such that the frame provides radial force during installation and later reduces that force as the materials resorb. The material selection and application, in combination with the frame design, can enable resorption of the bioresorbable materials reduce radial force as the prosthetic adequately integrates within the local anatomy, and further allowing for healing and adhesion of the implant to the native anatomy

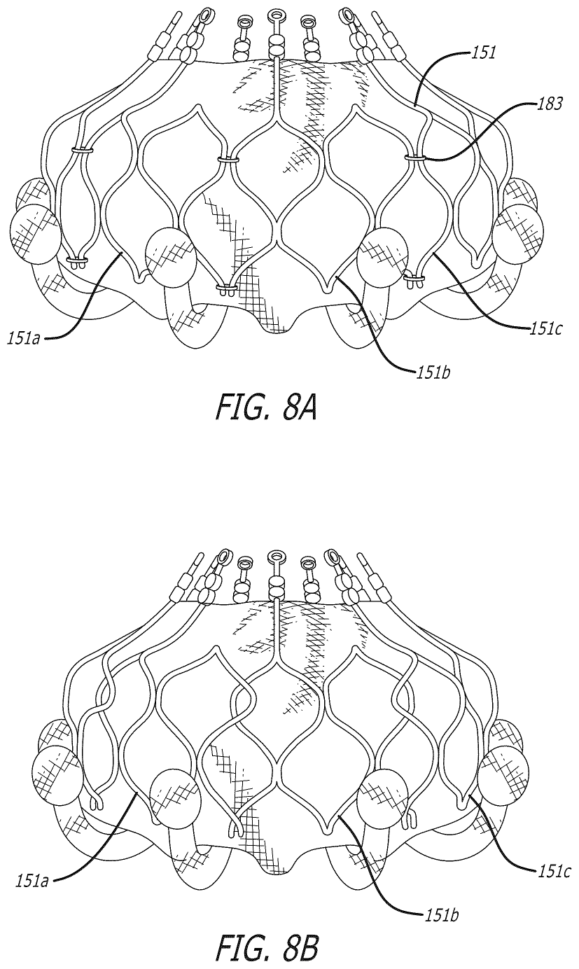

FIG. 8A provides an example of a frame 151 that can be constricted. In this example, frame 151 is divided into multiple segments (151a, 151b, 151c) that in combination yield a circular frame. Each frame segment is adjoined to its adjacent frame segments via bioresorbable connectors 183. Any bioresorbable connector capable of adjoining adjacent segments can be utilized, such as (for example) a small band (as shown in FIG. 8A), a knot, a hook, a rivet, a staple, or a column of a plurality of biocompatible elements (as shown in FIG. 2A). After installation at the site of implantation, bioresorbable connector 183 can resorb remove the restriction upon the multiple segments. The local anatomy at the site of implantation can provide constrictive forces, causing the segments to slide amongst one another such that they overlap, resulting in a constricted frame and valvular prosthetic (FIG. 8B). When installing the frame, it can be expanded via a balloon or a mechanical means, or is composed of a shape-memory material (e.g., nitinol).

A valvular prosthetic can comprise one or more of any of the bioresorbable components and/or elements described herein. Accordingly, the various described bioresorbable components can be combined in any way. In various valvular prosthetics, one or more of the following is combined: a frame having one or more bioresorbable elements, one or more bioresorbable anchors, one or more bioresorbable barbs, a bioresorbable inner skirt, a bioresorbable outer skirt, a fluffy bioresorbable outer skirt, one or more fluffy bioresorbable outer bands, a bioresorbable band for preventing expansion of a frame, and a frame comprising segments that are linked via a bioresorbable connector.

A valvular prosthetic having one or more bioresorbable components can be loaded within a transcatheter delivery device. The valvular prosthetic is crimped and loaded within a catheter such that the prosthetic can be delivered via the transcatheter approach to a site of implementation. Any appropriate transcatheter delivery system can be employed, such as one described in US Patent Publication No. 2017/0231756, the disclosure of which is incorporated herein by reference it its entirety. Accordingly, a delivery system can comprise a transcatheter with a valvular prosthetic having one or more bioresorbable components and/or elements. The valvular prosthetic having one or more bioresorbable components/elements and the delivery system can be sterilized and stored.

In another aspect, a frame is constructed for improved crimping. A typical frame is constructed such that cells formed by frame struts have a generally symmetrical shape. Symmetry, however, can cause issues with crimping due to equivalent struts and connectors having a symmetrical design each trying to crimp inward at the same time with same force. Issues with symmetrical designs include non-uniform strut twisting leading to oval and saddle-shaped frame configurations during crimping and deployment.

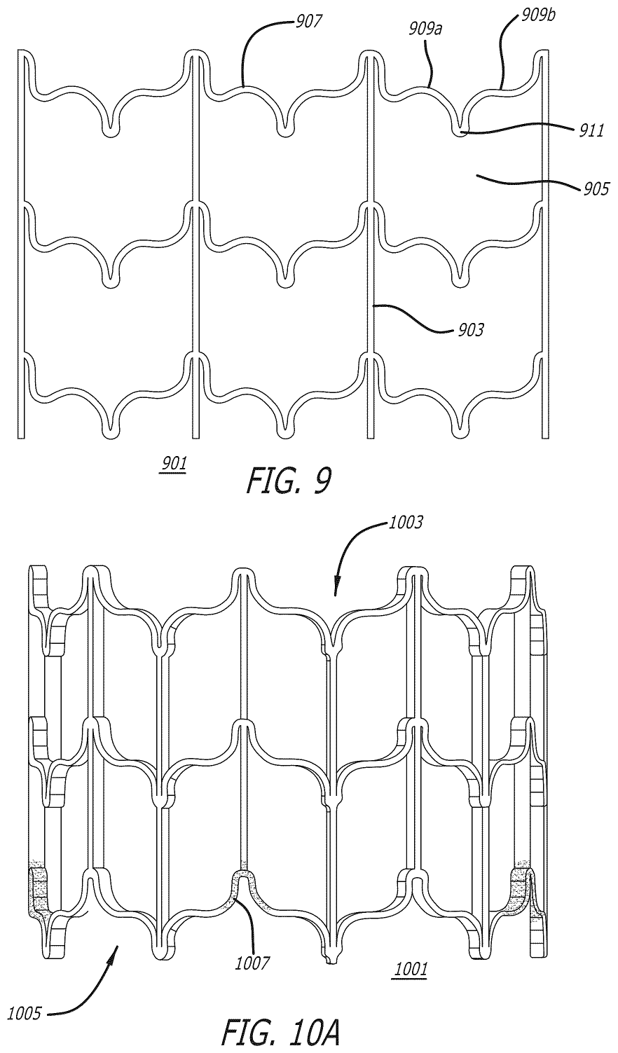

Provided in FIG. 9 is a frame design in which each cell comprises an asymmetrical shape. It has been discovered that an asymmetrical struts can provide improved crimping ability. Frame 901 is comprised of interconnected struts 903 that form a number of cells 905. For each cell, at least one strut 907 that extends in a direction along the circumference of the frame has an asymmetrical design that lacks reflection symmetry across the central midline of the strut. The lack of reflection symmetry can be formed by incongruent length and/or curvature on each side of the midline. Struts 907 each have two curved portions (909a and 909b) that meet in a middle apex 911, marking the central midline of the asymmetrical struts. Curved portion 909a has a greater radius and length than curved portion 909b. This asymmetrical design allows one side of the cell to crimp or close circumferentially before the other side of the cell and allows for predictable loading by forcing all apices to the same direction during the crimping process. Similarly, this asymmetric bias provides for more predictable valve crimping and expansion, reducing randomness. Although FIG. 9 depicts frame cells with asymmetrical curved portions, various other asymmetrical designs can be utilized to improve crimping, including the use of multiple struts to yield an asymmetrical design along the circumference of the frame

Frames with asymmetrical cells and struts can be utilized as stents or within a valvular prosthetic. When in the crimped formation, the frame can be packed into a transcatheter delivery device such that it can be used in a transcatheter procedure. Accordingly, a delivery system can comprise a transcatheter with a crimped frame having one or more cells with an asymmetrical design. The crimped frame having one or more cells with an asymmetrical design and/or the delivery system can be sterilized and stored.

When delivered to the site, any method of expanding the frame can be utilized, including expanding via a balloon, mechanical expansion, or utilization of a shape-memory material (e.g., nitinol). Accordingly, in some implementations, the frame is crimped with a balloon or other means for mechanical expansion.

In another aspect, a prosthetic device is provided with features that reduce blood stagnation and thereby reduce the likelihood of clotting. In a prosthetic heart valve, blood flow can stagnate in the space between the leaflets and the surrounding luminal wall. Stagnant blood can cause clotting, increase embolism risk and other issues and can limit the lifetime of a valve. Thus, there is a need for solutions to improve blood flow and/or washout on the outflow side of the leaflets.

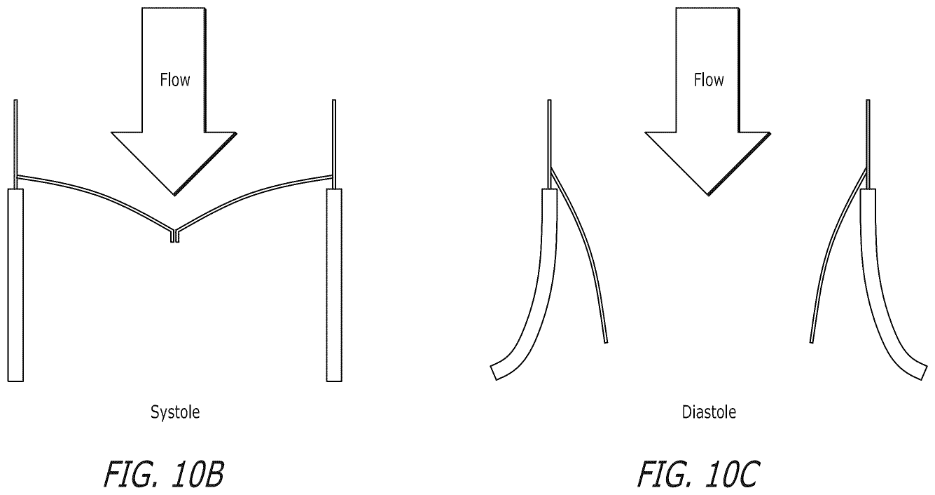

One implementation found to improve stagnant blood flow is to utilize a valvular prosthetic with a flared-out outflow end. However, there may be a need to deliver and/or install the valvular prosthetic straight-lined shape on the outflow end, which can improve delivery by reducing interactions with the delivery system. FIGS. 10A to 10C provide a system for providing a valvular frame with a straight-lined shape on the outflow end for delivery and installation that converts its outflow end to flare outwards. FIG. 10A depicts an example of a valvular frame 1001 for use in valvular prosthetic. Frame 1001 has an inflow side 1003 and outflow side 1005. Frame 1001 further includes a number of bioresorbable elements 1007 at or near outflow side 1005. Bioresorbable elements 1007 will resorb over time, yielding frame 1001 with a segmented outflow side 1005 with an ability to flex. After resorbing, outflow side 1005 would be able to flex outward when the local blood pressure and flow is high. This would result in a frame that would flexes outward during diastole (see FIGS. 10B and 10C), resulting in valvular prosthetic with a flared-out outflow end to help case outward flow and thus prevent blood flow stagnation.

Various implementations of bioresorbable elements can be utilized to yield a flared outflow end. In some implementations, a bioresorbable element is provided at the outflow end of every other vertical strut. In some implementations, a bioresorbable element is provided at the outflow end of every third vertical strut. In some implementations, a bioresorbable element is provided at the outflow end of every fourth vertical strut. In some implementations, a bioresorbable element is provided at the outflow end of every fifth vertical strut. Various implementations can be combined, such as (for example) an implementation having, for a first set of struts, a bioresorbable element every other strut, and for second set of struts, a bioresorbable element every third strut.

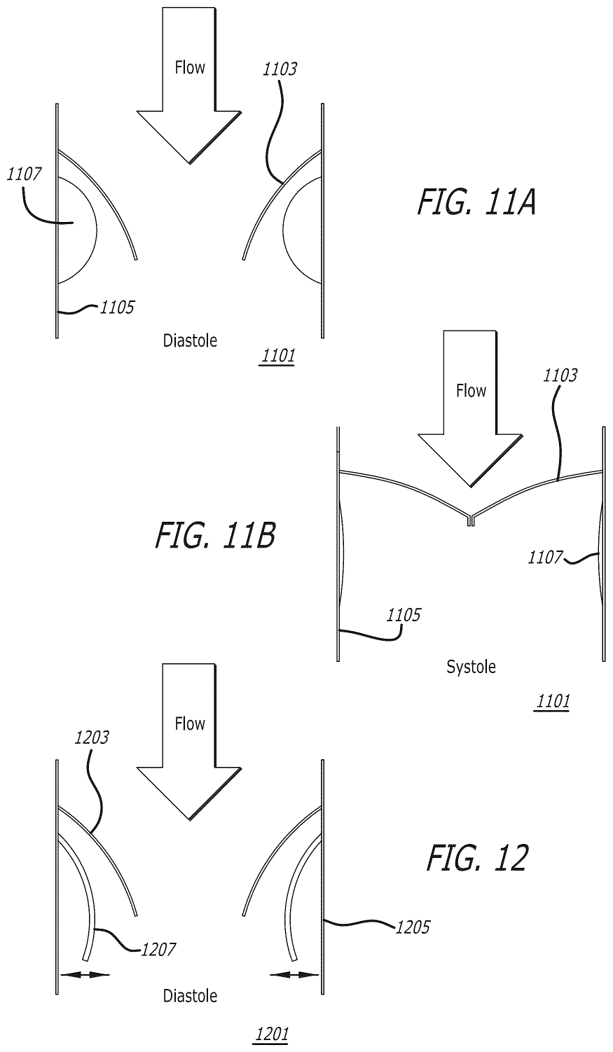

FIGS. 11A and 11B provide an example of a valvular prosthetic to improve stagnant blood flow utilizing a gap-filling member, such as an inflatable bag. Valvular prosthetic 1101 has a set of leaflets 1103 and inner luminal wall 1105. Underneath leaflets 1103 and attached to inner luminal wall 1105 are a set of one or more gap filling members, which displace blood and thereby reduce blood stagnation. In one example, inflatable bags 1107 are filled with a compressed fluid component (e.g., compressed liquid or gaseous component) that changes volume based on pressure. When the valve is closed there is no flow through the lumen of the valve and the set of inflatable bags 1107 can rest in an essentially unexpanded state (FIG. 11B). When the leaflets 1103 open to allow blood flow through the valve, the flow is increased and inflatable bags 1107 to expand with outward flow, creating an hourglass like shape in the lumen of the valve (FIG. 11A). The inflation of the bags can propel stagnating blood along luminal wall 1105 towards the center of valvular prosthetic 1101 and through the outflow end, assisted by the hourglass-like shape of the valvular lumen. The inflatable bags can be composed of any biocompatible flexible material, such as (for example) PET.

FIG. 12 provides an example of a valvular prosthetic to improve stagnant blood flow utilizing free-flowing sheet. Valvular prosthetic 1201 has a set of leaflets 1203 and inner luminal wall 1205. Underneath leaflets 1203 and attached to inner luminal wall 1205 are a set of one or more free-flowing sheet 1207 that have one edge attached to the luminal wall. During systole when local pressure and flow are low, the set of free-flowing sheets 1207 can rest along or near luminal wall 1205 (FIG. 12). When the flow and pressure are increased the leaflets 1203 open to allow blood flow through and the free edge of set of sheets 1207 to move with outward flow (FIG. 12). The movement of the sheets can propel any stagnating blood along luminal wall 1205 towards the center of valvular prosthetic 1201 and through the outflow end.

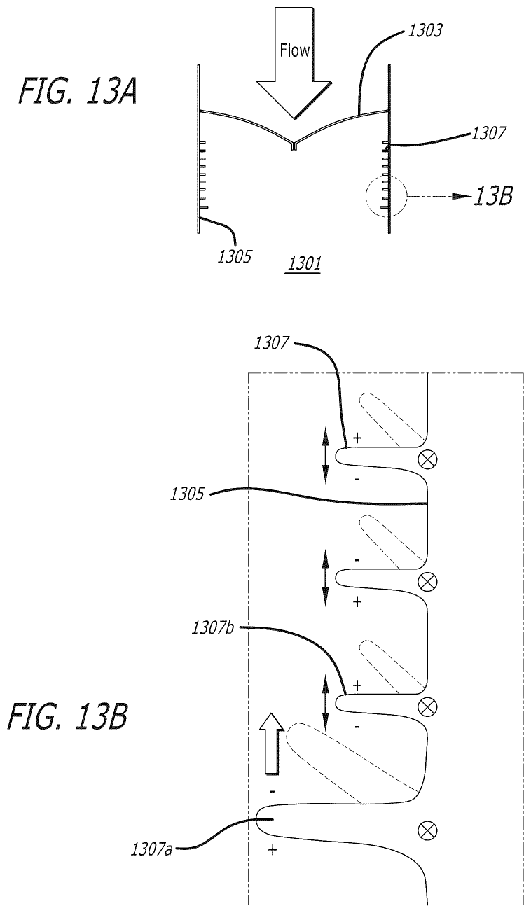

FIGS. 13A and 13B provide an example of a valvular prosthetic to improve stagnant blood flow utilizing movable microprotrusions. Valvular prosthetic 1301 has a set of leaflets 1303 and inner luminal wall 1305. Underneath leaflets 1303 and attached to inner luminal wall 1305 are a set of microprotrusions 1307 that protrude away the luminal wall. In one example, the microprotrusions are magnetically drive wherein each has a positive magnetic pole and negative magnetic pole at the microprotrusion tip. Each microprotrusion can flex in a direction toward the leaflets. The set of magnetically driven microprotrusions 1307 can include one or more larger driver microprotrusions 1307a, which can stimulate the flexing of the rest of the set of microprotusions within a linearly alignment in the direction of flow. Further, each microprotrusion tip has a particular pole alignment such that each the pole face of each tip is the same charge as the adjacent tip pole face. For instance, as shown in FIG. 13B, the larger driver microprotrusion tip 1307a has negative-positive pole tip and adjacent tip 1307b has positive-negative pole tip such that negative pole face of microprotrusion tip 1307a is directly adjacent to the negative pole face of microprotrusion tip 1307b. Thus, when the driver microprotrusion tip 1307a flexes toward microprotrusion tip 1407b it pushes microprotrusion tip 1307b to flex toward the leaflets. Each adjacent microprotrusion tip with opposite polarity along the linear alignment allows the driver microprotrusion tip to sequentially induce the flexing of each microprotrusion. The flexing of the microprotrusion can propel any stagnating blood along luminal wall 1305 towards the center of valvular prosthetic 1301 and through the outflow end.

In another aspect, frames and catheters are provided to prevent or limit strut torsion. An issue with tubular frames is that struts tend to contort and twist when loaded into a sheath. This is especially true for struts in a less dense portion of the frame, which have more space to allow the torsion to occur. Because the torsion can damage the frame and/or create issues during deployment and installation, it is best practice to load the frame into the sheath such that no torsion occurs. Generally, to prevent strut torsion, frames are crimped with a device that individually restrains each strut such that it cannot contort. This device, however, is generally only used during the initial crimping and loading of the frame. Its use is limited to only extracorporeal loading and generally not available bedside.

Clinicians desire to have the ability to proximally reload the structural frame back into the sheath after distally advancing the frame at a site of installation during transcatheter procedures. The deployment and installment of the frame requires extreme precision at the site of installation within the patient. Because reloading of structural frames can cause strut torsion, clinicians only have a single opportunity to deploy and install frames, which can be difficult when relying on cchography or radiography imaging. While at the site of installation, clinicians desire to proximally reload the frame back into the sheath such that they can adjust and reposition the sheath, and then distally advance and deploy the frame again at the repositioned site. This would allow better installation procedures as it would not require a perfectly precise deployment with the first attempt. When deployed, the frame can be expanded via a balloon, mechanical expansion, or utilization of a shape-memory material (e.g., nitinol) to yield a self-expanding frame.

To prevent torsion of struts during crimping and loading (and reloading) of a valvular frame into a sheath, a frame can comprise one or more anti-torsion elements on a set of struts. An anti-torsion element is a small protrusion extending laterally from the strut in a direction consistent with the frame circumference. The anti-torsion element prevents torsion by providing a laterally wider contour that does not allow the strut to contort or twist as it is loaded into a sheath. An anti-torsion element can be apart of the frame design such that when the frame is fabricated with the elements. Alternative, anti-torsion elements can be attached onto a frame, which can be attached and secured to the strut via a means of attachment (for example, via rivets, screws, adhesive, or snapping into place).

In some implementations, an anti-torsion element laterally extends a length that prevents contortion when loading, as can be determined by the lateral width and radial depth of the strut. In some implementations, an anti-torsion element laterally extends a length such that the protrusion abuts or nearly abuts an adjacent strut. In some implementations, an anti-torsion element laterally extends a length between about 1.1 to 5 times the lateral width of the strut. In various implementations, the anti-torsion element laterally extends a length: about 1.1 times the lateral width of the strut, about 1.5 times the lateral width of the strut, about 2 times the lateral width of the strut, about 2.5 times the lateral width of the strut, about 3 times the lateral width of the strut, about 3.5 times the lateral width of the strut, about 4 times the lateral width of the strut, about 4.5 times lateral width of the strut, or about 5 times the lateral width of the strut. In some implementations, an anti-torsion element has the same (or near same) radial depth as the strut. In some implementations, an anti-torsion element has vertical length (i.e., length parallel with the longitudinal axis of the strut) that is a fraction of the length of the strut. The vertical length can vary, but should not be so long to prevent the flexibility of the strut and the expandability of the frame. In some implantations, the vertical length of an anti-torsion element is between about 2% to 20% of the length of the strut. In various implementations, the vertical length of an anti-torsion element is: about 2% of the length of the strut, about 2.5% of the length of the strut, about 3% of the length of the strut, about 5% of the length of the strut, about 10% of the length of the strut, or about 20% of the length of the strut. An anti-torsion element can have any shape.

One or more anti-torsion elements can be provided on each strut of the frame, or on each strut a set of struts. In some implementations, one or more anti-torsion elements are provided on struts that are within a less dense portion of the frame (as determined by density along the frame circumference). Density can be determined by the abutment (or near abutment) of struts in the crimped state. When crimped, adjacent struts that are abutting or within a distance that prevents the ability of the strut to twist may not need an anti-torsion element because this density prevents strut cotorsion. The distance to prevent twisting can be determinable by the lateral width and radial depth of the strut. Further, high lateral density may not allow for the addition of anti-torsion elements that would widen the lateral contour of the strut.

An anti-torsion can further comprise other components. In some implementations, an anti-torsion element comprises a marker for visualization via echography, radiography, or any other visualization technique for monitoring during transcatheter procedures. In some implementations, an aperture is provided within an anti-torsion element, which can be utilized as a marker for visualization.

Alternatively, or in addition to a frame having an anti-torsion element, a sheath can be designed such that struts of the frame are unable to twist and contort. Sheaths are generally tubular in design having an inner lumen. The inner surface of the sheath (i.e., the surface of the luminal interior of the sheath) has a circular contour. To prevent twisting and contorting, the inner surface of the sheath can have a polygonal contour instead of the traditional circular contour. The flat contour of each side of the polygon reduces the amount of space between the struts and the inner surface of the sheath, and thus the strut does not have the space to twist and contort.

Valvular frames often comprise a repeated pattern of struts and cells, yielding a plurality of repeated columnar segments (e.g., each segment extends along the proximal-distal axis). The number of columnar segments can vary, but are generally between 5 and 15 columnar segments, as dependent on the frame design. An inner surface of sheath can be contoured with a polygon to match the columnar segments of the frame. For instance, the inner surface contour of the sheath can be a polygon having a number sides matching the number of columnar segments of the frame. Accordingly, in various implementations, the inner contour of the sheath is: a pentagon for a frame having five columnar segments, a hexagon for a frame having six columnar segments, a septagon for a frame having seven columnar segments, an octagon for a frame having eight columnar segments, a nonagon for a frame having nine columnar segments, a decagon for a frame having ten columnar segments, a hendecagon for a frame having eleven columnar segments, a dodecagon for a frame having twelve columnar segments, a tridecagon for a frame having thirteen columnar segments, a tetradecagon for a frame having fourteen columnar segments, or a pentadecagon for a frame having fifteen columnar segments.

In some implementations to prevent strut torsion, when a crimped frame is within a sheath comprising an inner surface with a polygonal contour, a strut is within close proximity to a side of the polygon. In some implementations, when a crimped frame is within a sheath comprising an inner surface with a polygonal contour, a strut abuts or nearly abuts a side of the polygon. The measurement of strut width and near abutment distance is to be measured at the same location of the strut.

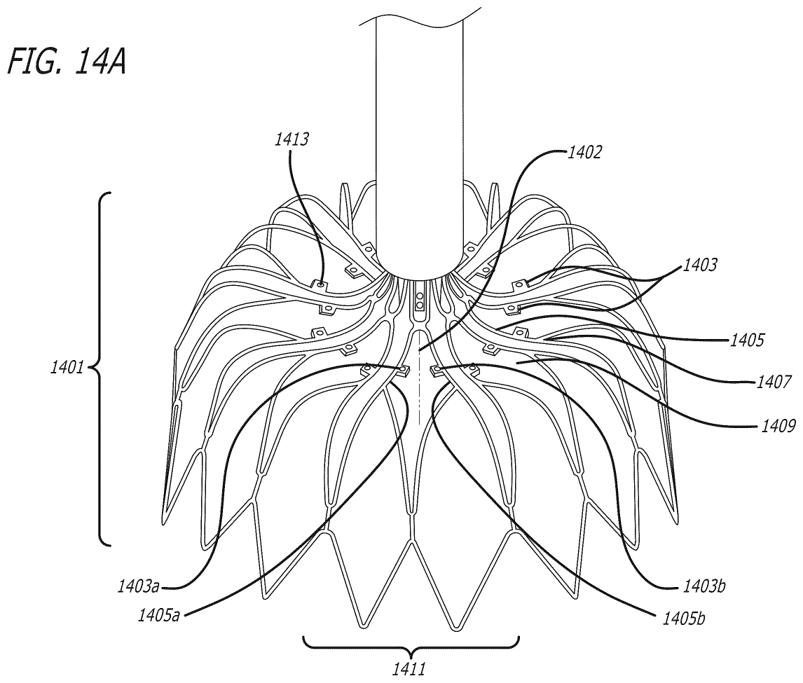

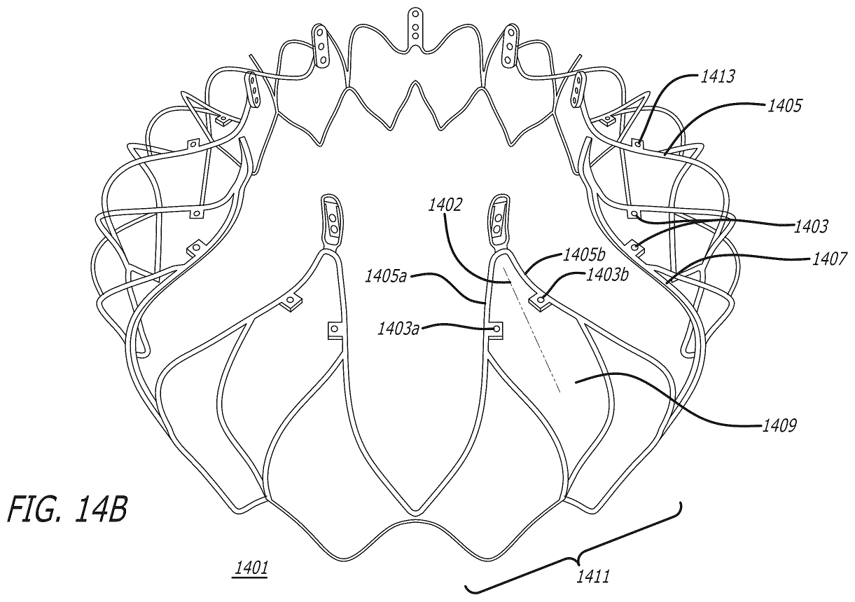

Provided in FIGS. 14A and 14B is an example of a frame having a plurality of anti-torsion elements. Frame 1401 comprises a plurality anti-torsion elements 1403. Frame 1401 comprises a plurality of struts 1405 that connect at a plurality of connecting points 1407 and forming a plurality of cells 1409, each cell perimeter comprising two or more connected struts. The plurality of struts 1405 form nine columnar segments, each columnar segment 1411 laterally adjacent to another columnar segment and in totality form the circumference.

Each columnar segment 1411 comprises three cells, with two cells at the distal end of the frame and a single cell at the proximal end. As can be readily appreciated, when crimped, the distal end will have a greater density of struts along the lateral circumference. Because of the lack of density of struts at the proximal end, these struts (e.g., strut 1405a and strut 1405b) have a propensity to twist and contort when crimped. To mitigate torsion on these struts, each strut at the proximal end comprises an anti-torsion element 1403.

As depicted, each anti-torsion element 1403 extends into a proximal cell, but an anti-torsion element could extend away from a cell or in any lateral direction off a strut. In some implementations, two or more anti-torsion elements extend towards the same latitudinal axis, each anti-torsion element is provided on a different longitudinal axis, which can prevent the anti-torsion elements from coming into contact when the frame is crimped. For example, anti-torsion element 1403a and anti-torsion element 1403b each extend toward the same latitudinal axis 1402. Anti-torsion element 1403a extends off of strut 1405a along a longitudinal axis that is proximal to a longitudinal that anti-torsion element 1403b extends along, which extends from strut 1405b. When frame 1401 is crimped, anti-torsion element 1403a and anti-torsion element 1403b will not contact.

Each anti-torsion element 1403 comprises an aperture 1413, which can be utilized for visualizing the element and frame 1401 via a visualization technique for monitoring deployment of the frame. Each anti-torsion element 1403 is depicted as a quadrilateral, but any shape can be utilized.

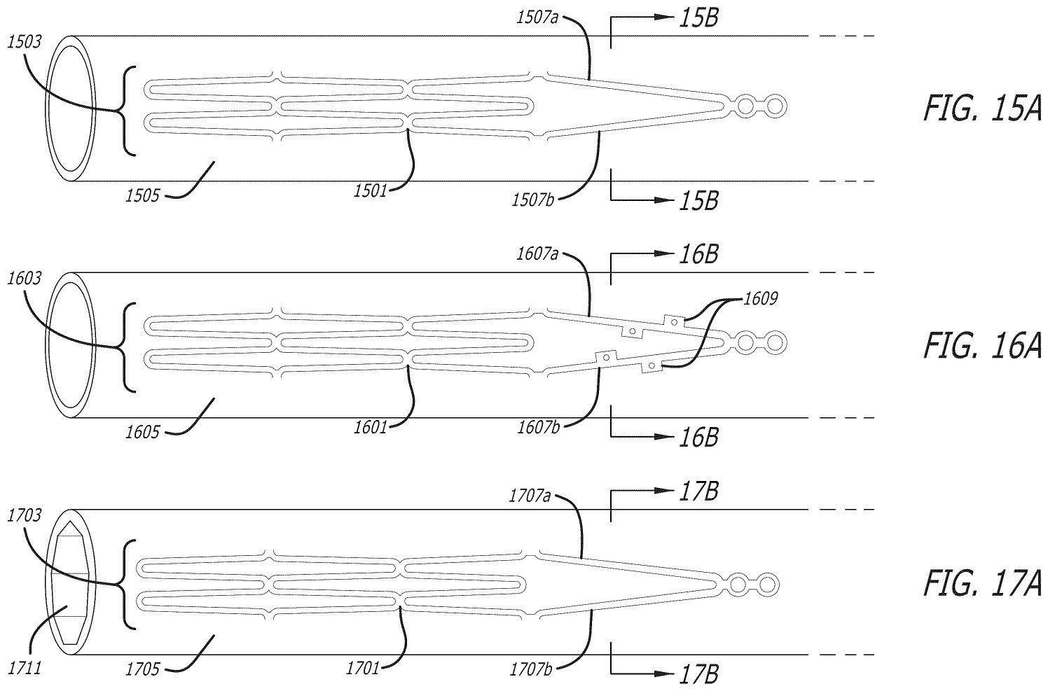

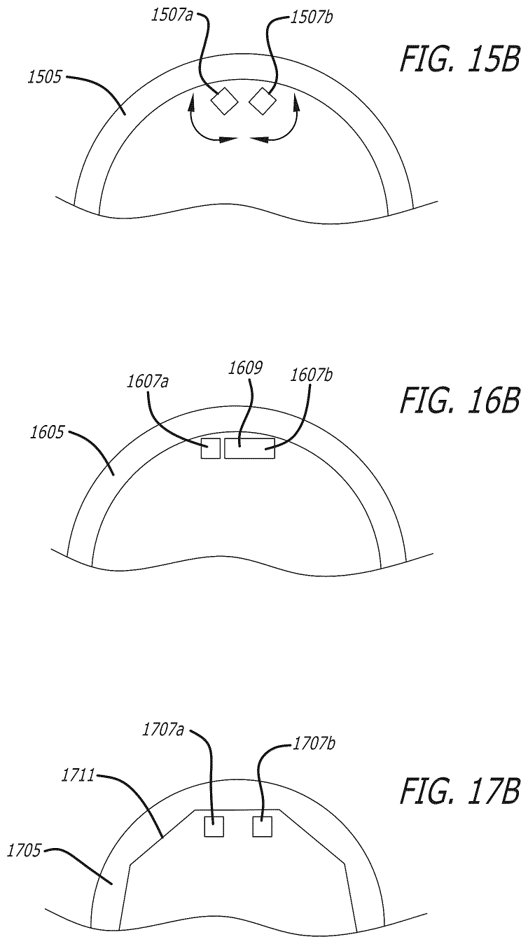

FIGS. 15A and 15B depict an example of a frame crimped within a sheath. The frame comprises a series of columnar segments but the figures depict only a single columnar segment for the sake of clarity and explanation. Frame 1501 comprises a columnar segment 1503 within sheath 1505. Strut 1507a and strut 1507b are located in a section of frame 1501 that is lower in density of struts with enough space to twist and contort, and thus these struts have a propensity to twist and contort when the frame is crimped and loaded into the sheath (see arrows 1508 in FIG. 15B).

FIGS. 16A and 16B depict an example of a frame comprising anti-torsion elements crimped within a sheath. The frame comprises a series of columnar segments but the figures depict only a single columnar segment for the sake of clarity and explanation. Frame 1601 comprises a columnar segment 1603 within sheath 1605. Strut 1607a and strut 1607b are located in a section of frame 1601 that is lower in density of struts with enough space to twist and contort. To mitigate twisting and contortion, strut 1607a and strut 1607b comprise a set of anti-torsion elements. As depicted, each of strut 1607a and strut 1607b comprise two anti-torsion elements, but it should be understood that a set can be one or more, and can vary in dimensions and shape, as described herein. Anti-torsion elements 1609 restrict the ability of strut 1607a and strut 1607b to twist and contort, reducing the torsion on the struts (FIG. 16B).

FIGS. 17A and 17B depict an example of a frame crimped within a sheath comprising an inner surface having a polygonal shape. The frame comprises a series of columnar segments but the figures depict only a single columnar segment for the sake of clarity and explanation. Frame 1701 comprises a columnar segment 1703 within sheath 1605. Strut 1707a and strut 1707b are located in a section of frame 1701 that is lower in density of struts with enough space to twist and contort. To mitigate twisting and contortion, the inner surface 1711 of a sheath 1701 is polygonal. As shown, columnar segment 1703 abuts or nearly abuts a side of the polygonal contour. Polygonal contour of inner surface 1711 restricts the ability of strut 1707a and strut 1707b to twist and contort, reducing the torsion of the struts (FIG. 16B).

In some implementations, a frame comprises anti-torsion elements (e.g., FIG. 16A) and is crimped and loaded into a catheter comprising an inner face with a polygonal contour (e.g., FIG. 17B).

In another aspect, an improved manufacturing procedure is provided wherein a cover may be applied over a frame in a more efficient and economical manner. Common issue with manufacturing of valvular frame cover is that it can be difficult to wrap and attach a cover around the frame. Covers are generally a sheet of fabric, tissue, film, or some other flattened material and frames are typically tubular in shape with numerous circumferences of varying length. When wrapping and securing the cover to the frame, excess cover can bunch up and require further trimming and fitting. To solve this issue, a cover can be precut prior to assembly. Precut covers can be repeated and scaled up to industrial manufacturing levels, facilitating the fabrication of frames with covers.

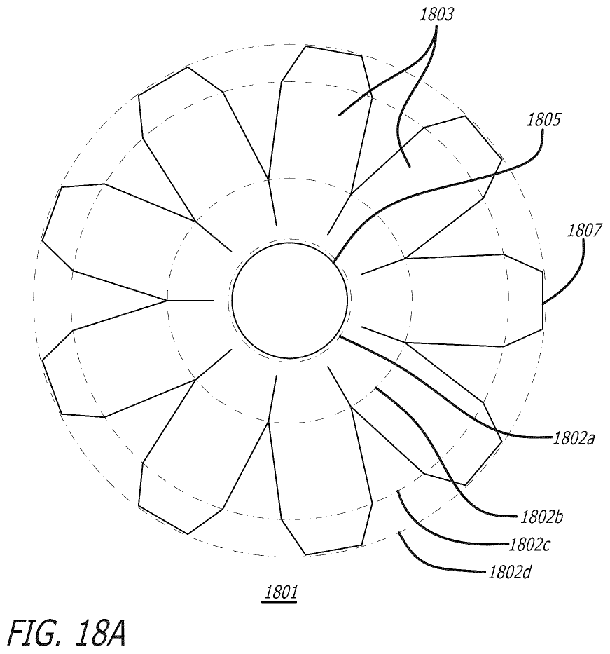

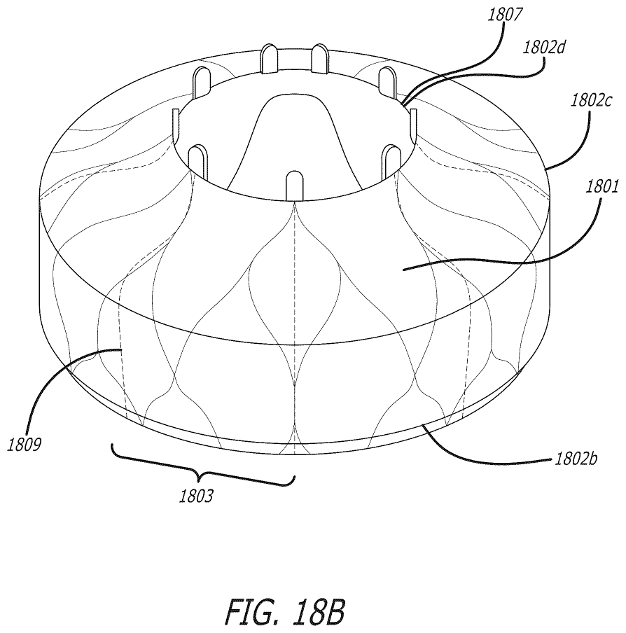

Provided in FIG. 18A is an example of a precut cover for assembly onto a frame. Cover 1801 comprises a plurality of segments 1803. The number of segments can vary and depend on the frame design. Cover 1801 is for a frame comprising nine columnar segments, and in accordance with some implementations, the cover comprises the same number of segments as the columnar segments of the frame. Segments 1803 are cut in a manner to adequately cover each columnar segment of the frame. In this example, each segment 1803 is in connection with adjacent segments at a first end 1805 of cover 1801 (i.e., the first end is one of the proximal end or the distal end). At a second end 1807 opposite of the first end, each segment 1803 is not connected from adjacent segments, such that the cover 1801 is precut in a shape resembling a flower. The lateral edges of each segment 1803 comprise a portion that is connected at or near the first end and a portion that is not connected that is not at or near the first end (e.g., midportion and portion at or near the second end). As shown, the majority of each lateral edge each segment 1803 is not connected from the adjacent segments.

Cover 1801 is for a tubular frame that comprises a plurality of circumference lengths along the proximal-distal axis. FIG. 18A depicts four circumference lengths (1802a, 1802b, 1802c, and 1802d) of the frame. Accordingly, each segment 1803 is cut to a specification with the lateral edges of each segment 1803 contours to yield a segment width to match the plurality of circumferences of the frame. As depicted, first end 1805 is cut to a size matching circumference length 1802a. Each segment 1803 expands laterally from first circumference length 1802a to second circumference length 1802b and then maintains a similar lateral width until third circumference 1803c. The width of each segment 1803 decreases laterally from third circumference length 1802c to fourth circumference length 1802d, which is the second end of the cover. Each lateral edge can be utilized to suture the cover onto the frame

FIG. 18B provides the example cover of FIG. 18A attached onto a frame. Cover 1801 is fit onto the frame, overlaying each segment 1803 on a columnar segment of the frame. Each segment 1803 is sewn with each of its adjacent segments along its lateral side via stitching 1809, yielding a plurality of scams. Accordingly, the lateral edges of each segment 1803 is connected to the lateral edges of each adjacent section at first end 1805 by the precut flower shape (see FIG. 18A) and is connected to the lateral edges of each adjacent section at second end 1807 by a means of attachment, such as the stitching 1809. The majority of the lateral edges of each columnar segment is connected to the lateral edges of each adjacent section by a means of attachment, such as the stitching 1809. Although stitching is shown, any many means of attachment can be utilized such as (for example) stitching, staples, and an adhesive.

The following examples are included within the scope of the invention.

Example 1. A frame for use in a valvular prosthetic, comprising a plurality of interconnected struts that form a tubular frame, and a plurality of bioresorbable elements within a subset of the struts that form the tubular frame.

Example 2. The frame as in example 1, wherein the plurality of interconnected struts comprises shape-memory material that provides a radial force.

Example 3. The frame as in example 2, wherein resorption of the bioresorbable elements results in weakening the radial force of the tubular frame.

Example 4. The frame as in example 1, 2, or 3, wherein at least a subset of the plurality of bioresorbable elements are located at an interconnection point of two or more struts.

Example 5. The frame as in any one of examples 1-4, wherein the tubular frame further comprises a plurality of appendages that extend away from the tubular frame; and wherein each appendage of the plurality of appendages is formed by a subset of the plurality of interconnected struts.

Example 6. The frame as in example 5, wherein at least a subset of the plurality of bioresorbable elements are located within one or more appendages of the plurality of appendages.

Example 7. The frame as in any one of examples 1-6, wherein the tubular frame is crimped.

Example 8. The frame as in example 7, wherein the tubular frame is within a catheter of a transcatheter delivery system.

Example 9. The frame as in any one of examples 1-8 further comprising an inner skirt attached to the tubular frame.

Example 10. The frame as in example 9, wherein the inner skirt is bioresorbable.

Example 11. The frame as in any one of examples 1-10 further comprising a set of leaflets within the tubular frame; wherein the set of leaflets are attached to the tubular frame or attached to the inner skirt.

Example 12. The frame as in any one of examples 1-11 further comprising an outer skirt attached to the tubular frame.

Example 13. The frame as in example 12, wherein the outer skirt is bioresorbable.

Example 14. The frame as in any one of examples 1-13 further comprising a fluffy bioresorbable band that encircles the tubular frame.

Example 15. The frame as in any one of examples 1-14 further comprising an anchoring system attached to the tubular frame.

Example 16. The frame as in example 15, wherein the anchoring system comprises a bioresorbable portion.

Example 17. The frame as in example 16 further comprising a set of bioresorbable barbs attached to the tubular frame or attached to the anchoring system.

Example 18. The frame as in any one of examples 1-17, wherein the frame is sterilized and packaged.

Example 19. A valvular prosthetic, comprising: a plurality of interconnected struts that form a tubular frame; and an anchoring system attached to the tubular frame, the anchoring system comprising a plurality of anchoring arms having bioresorbable anchor tips.

Example 20. The valvular prosthetic as in example 19, wherein resorption of at least one of the bioresorbable anchor tips results in a reduction of intrusion into local tissue at an implantation site when implanted.

Example 21. The valvular prosthetic as in example 19 or 20 further comprising a plurality of bioresorbable elements within a subset of the struts that form the tubular frame.

Example 22. The valvular prosthetic as in example 21, wherein at least a subset of the plurality of bioresorbable elements are located at an interconnection point of two or more struts.

Example 23. The valvular prosthetic as in example 21 or 22, wherein the tubular frame further comprises a plurality of appendages that extend away from the tubular frame; wherein each appendage of the plurality of appendages is formed by a subset of the plurality of interconnected struts; and wherein at least a subset of the plurality of bioresorbable portions are located within one or more appendages of the plurality of appendages.

Example 24. The valvular prosthetic as in any one of examples 19-23, wherein the valvular prosthetic is crimped.

Example 25. The valvular prosthetic as in example 24, wherein the crimped valvular prosthetic is within a catheter of a transcatheter delivery system.

Example 26. The valvular prosthetic as in any one of examples 19-25 further comprising an inner skirt attached to the tubular frame.

Example 27. The valvular prosthetic as in example 26, wherein the inner skirt is bioresorbable.

Example 28. The valvular prosthetic as in any one of examples 19-27 further comprising a set of leaflets within the tubular frame; wherein the set of leaflets are attached to the tubular frame or attached to the inner skirt.

Example 29. The valvular prosthetic as in any one of examples 19-28 further comprising an outer skirt attached to the tubular frame.

Example 30. The valvular prosthetic as in example 29, wherein the outer skirt is bioresorbable.

Example 31. The valvular prosthetic as in any one of examples 19-30 further comprising a fluffy bioresorbable band that encircles the tubular frame.

Example 32. The valvular prosthetic as in any one of examples 19-31 further comprising a set of bioresorbable barbs.

Example 33. The valvular prosthetic as in any one of examples 19-32, wherein the valvular prosthetic is sterilized and packaged.

Example 34. A valvular prosthetic, comprising: a plurality of interconnected struts that form a tubular frame; and an inner skirt attached to the tubular frame, the inner skirt is bioresorbable.

Example 35. The valvular prosthetic as in examples 34 further comprising a plurality of bioresorbable elements within a subset of the struts that form the tubular frame.

Example 36. The valvular prosthetic as in example 35, wherein at least a subset of the plurality of bioresorbable elements are located at an interconnection point of two or more struts.

Example 37. The valvular prosthetic as in example 35 or 36, wherein the tubular frame further comprises a plurality of appendages that extend away from the tubular frame, wherein each appendage of the plurality of appendages is formed by a subset of the plurality of interconnected struts, and wherein at least a subset of the plurality of bioresorbable elements are located within one or more appendages of the plurality of appendages.

Example 38. The valvular prosthetic as in any one of examples 34-37, wherein the valvular prosthetic is crimped.

Example 39. The valvular prosthetic as in example 38, wherein the crimped valvular prosthetic is within a catheter of a transcatheter delivery system.

Example 40. The valvular prosthetic as in any one of examples 34-39 further comprising a set of leaflets within the tubular frame; wherein the set of leaflets are attached to the tubular frame or attached to the inner skirt.

Example 41. The valvular prosthetic as in any one of examples 34-40 further comprising an outer skirt attached to the tubular frame.

Example 42. The valvular prosthetic as in example 41, wherein the outer skirt is bioresorbable.

Example 43. The valvular prosthetic as in any one of examples 34-42 further comprising a fluffy bioresorbable band that encircles the tubular frame.

Example 44. The valvular prosthetic as in any one of examples 34-43 further comprising an anchoring system attached to the tubular frame.

Example 45. The valvular prosthetic as in example 44, wherein the anchoring system comprises a bioresorbable portion.

Example 46. The valvular prosthetic as in example 45 further comprising a set of bioresorbable barbs attached to the tubular frame or attached to the anchoring system.

Example 47. The valvular prosthetic as in any one of examples 34-46, wherein the valvular prosthetic is sterilized and packaged.

Example 48. A valvular prosthetic, comprising: a plurality of interconnected struts that form a tubular frame; and an outer skirt attached to the tubular frame, the outer skirt is bioresorbable.

Example 49. The valvular prosthetic as in example 48, wherein the outer skirt is a fluffy bioresorbable outer skirt.

Example 50. The valvular prosthetic as in example 48 or 49 further comprising a plurality of bioresorbable element within a subset of the struts that form the tubular frame.

Example 51. The valvular prosthetic as in example 50, wherein at least a subset of the plurality of bioresorbable elements are located at an interconnection point of two or more struts.

Example 52. The valvular prosthetic as in example 50 or 51, wherein the tubular frame further comprises a plurality of appendages that extend away from the tubular frame; wherein each appendage of the plurality of appendages is formed by a subset of the plurality of interconnected struts; and wherein at least a subset of the plurality of bioresorbable elements are located within one or more appendages of the plurality of appendages.

Example 53. The valvular prosthetic as in any one of examples 48-52, wherein the valvular prosthetic is crimped.

Example 54. The valvular prosthetic as in example 53, wherein the crimped valvular prosthetic is within a catheter of a transcatheter delivery system.

Example 55. The valvular prosthetic as in any one of examples 48-54 further comprising an inner skirt attached to the tubular frame.

Example 56. The valvular prosthetic as in example 55, wherein the inner skirt is bioresorbable.

Example 57. The valvular prosthetic as in any one of examples 48-56 further comprising a set of leaflets within the tubular frame; wherein the set of leaflets are attached to the tubular frame or attached to the inner skirt.

Example 58. The valvular prosthetic as in any one of examples 48-57 further comprising an anchoring system attached to the tubular frame.

Example 59. The valvular prosthetic as in example 58, wherein the anchoring system comprises a bioresorbable portion.

Example 60. The valvular prosthetic as in example 59 further comprising a set of bioresorbable barbs attached to the tubular frame or attached to the anchoring system.

Example 61. The valvular prosthetic as in any one of examples 48-60, wherein the valvular prosthetic is sterilized and packaged.

Example 62. A valvular prosthetic, comprising: a plurality of interconnected struts that form a tubular frame; and a fluffy bioresorbable band that encircles the tubular frame.

Example 63. The valvular prosthetic as in example 62 further comprising a plurality of bioresorbable elements within a subset of the struts that form the tubular frame.

Example 64. The valvular prosthetic as in example 63, wherein at least a subset of the plurality of bioresorbable elements are located at an interconnection point of two or more struts.

Example 65. The valvular prosthetic as in example 63 or 64, wherein the tubular frame further comprises a plurality of appendages that extend away from the tubular frame; wherein each appendage of the plurality of appendages is formed by a subset of the plurality of interconnected struts; and wherein at least a subset plurality of bioresorbable elements are located within one or more appendages of the plurality of appendages.

Example 66. The valvular prosthetic as in any one of examples 62-65, wherein the valvular prosthetic is crimped.

Example 67. The valvular prosthetic as in example 66, wherein the crimped valvular prosthetic is within a catheter of a transcatheter delivery system.

Example 68. The valvular prosthetic as in any one of examples 62-67 further comprising an inner skirt attached to the tubular frame.

Example 69. The valvular prosthetic as in example 68, wherein the inner skirt is bioresorbable.

Example 70. The valvular prosthetic as in any one of examples 62-69 further comprising a set of leaflets within the tubular frame; wherein the set of leaflets are attached to the tubular frame or attached to the inner skirt.

Example 71. The valvular prosthetic as in any one of examples 62-70 further comprising an outer skirt attached to the tubular frame.

Example 72. The valvular prosthetic as in example 71, wherein the outer skirt is bioresorbable.

Example 73. The valvular prosthetic as in any one of examples 62-72 further comprising an anchoring system attached to the tubular frame.

Example 74. The valvular prosthetic as in example 73, wherein the anchoring system comprises a bioresorbable portion.

Example 75. The valvular prosthetic as in example 74 further comprising a set of bioresorbable barbs attached to the tubular frame or attached to the anchoring system.

Example 76. The valvular prosthetic as in any one of examples 62-75, wherein the valvular prosthetic is sterilized and packaged.

Example 77. A valvular prosthetic, comprising: a plurality of interconnected struts to form a tubular frame; an anchoring system attached to the tubular frame, and a set of bioresorbable barbs attached to the tubular frame or attached to the anchoring system.

Example 78. The valvular prosthetic as in example 77 further comprising a plurality of bioresorbable elements within a subset of the struts that form the tubular frame.

Example 79. The valvular prosthetic as in example 78, wherein at least a subset of the plurality of bioresorbable elements are located at an interconnection point of two or more struts.

Example 80. The valvular prosthetic as in example 78 or 79, wherein the tubular frame further comprises a plurality of appendages that extend away from the tubular frame; wherein each appendage of the plurality of appendages is formed by a subset of the plurality of interconnected struts; and wherein at least a subset of the plurality of bioresorbable elements are located within one or more appendages of the plurality of appendages.

Example 81. The valvular prosthetic as in any one of examples 77-80, wherein the valvular prosthetic is crimped.

Example 82. The valvular prosthetic as in example 81, wherein the crimped valvular prosthetic is within a catheter of a transcatheter delivery system.

Example 83. The valvular prosthetic as any of examples 77-82, wherein the anchoring system comprises a bioresorbable portion.

Example 84. The valvular prosthetic as in any one of examples 77-83 further comprising an inner skirt attached to the tubular frame.

Example 85. The valvular prosthetic as in example 84, wherein the inner skirt is bioresorbable.

Example 86. The valvular prosthetic as in any one of examples 77-85 further comprising a set of leaflets within the tubular frame; wherein the set of leaflets are attached to the tubular frame or attached to the inner skirt.

Example 87. The valvular prosthetic as in any one of examples 77-86 further comprising an outer skirt attached to the tubular frame.

Example 88. The valvular prosthetic as in example 87, wherein the outer skirt is bioresorbable.

Example 89. The valvular prosthetic as in any one of examples 77-88 further comprising a fluffy bioresorbable band that encircles the tubular frame.

Example 90. The valvular prosthetic as in any one of examples 77-89, wherein the valvular prosthetic is sterilized and packaged.

Example 91. A valvular prosthetic, comprising: a plurality of interconnected struts to form a tubular frame that is self-expanding; wherein the tubular frame comprises a shape-memory material; and a constricting bioresorbable band that encircles the self-expanding tubular frame.

Example 92. The valvular prosthetic as in example 91 further comprising an inner skirt attached to the tubular frame.

Example 93. The valvular prosthetic as in example 91 or 92 further comprising a set of leaflets within the tubular frame; wherein the set of leaflets is attached to the tubular frame or attached to the inner skirt.

Example 94. The valvular prosthetic as in example 91, 92, or 93 further comprising an outer skirt attached to the tubular frame.

Example 95. The valvular prosthetic as in any one of examples 91-94 further comprising an anchoring system attached to the tubular frame.

Example 96. The valvular prosthetic as in any one of examples 91-95, wherein the tubular frame is crimped.

Example 97. The valvular prosthetic as in example 96, wherein the valvular prosthetic is within a catheter of a transcatheter delivery system.

Example 98. The valvular prosthetic as in any one of examples 91-97, wherein the valvular prosthetic is sterilized and packaged.

Example 99. A valvular prosthetic, comprising a set of columnar segments, wherein each columnar segment comprises a plurality of interconnected struts, and a set of bioresorbable connectors, wherein the set of bioresorbable connectors connect the set of columnar segments to form a tubular frame, wherein each columnar segment is in connection with two adjacent columnar sections via one or more bioresorbable connectors of the set.

Example 100. The valvular prosthetic as in example 99 further comprising an inner skirt attached to the tubular frame.

Example 101. The valvular prosthetic as in example 99 or 100 further comprising a set of leaflets within the tubular frame; wherein the set of leaflets are attached to the tubular frame or attached to the inner skirt.

Example 102. The valvular prosthetic as in example 99, 100, or 101 further comprising an outer skirt attached to the tubular frame.

Example 103. The valvular prosthetic as in any one of examples 99-102 further comprising an anchoring system attached to the tubular frame.

Example 104. The valvular prosthetic as in any one of examples 99-103, wherein the tubular frame is crimped.

Example 105. The valvular prosthetic as in example 104, wherein the valvular prosthetic is within a catheter of a transcatheter delivery system.

Example 106. The valvular prosthetic as in any one of examples 99-105, wherein the valvular prosthetic is sterilized and packaged.

Example 107. A frame for use in a valvular prosthetic, comprising: a plurality of interconnected struts that form a tubular frame having a plurality of cells; wherein at least a subset of the plurality of cells is asymmetrical.

Example 108. The frame of example 107, wherein the asymmetry of each cell of the subset of the plurality of cells that is asymmetrical is formed by at least one cross strut that is asymmetrical.

Example 109. The frame of example 108, wherein the asymmetry of the at least one cross strut is formed by two curved portions that meet in a central apex, wherein a first curved portion of the two curved portions has a greater radius and length than a second curved portion of the two curved portions.

Example 110. A frame for use in a valvular prosthetic, comprising: a plurality of interconnected struts that form a tubular frame, the tubular frame having an inflow end and an outflow end; and a plurality of bioresorbable elements within a subset of the struts that form the tubular frame, wherein the bioresorbable elements are at or near the outflow side.

Example 111. The frame of example 110, wherein resorption of the bioresorbable elements are at or near the outflow side results in a segmented outflow end with an ability to flex.

Example 112. The frame of example 111, wherein the frame is configured such that when the frame experiences a liquid flow and pressure through the frame, the segmented outflow end is capable of flexing outward to convert the outflow end from a slight-line shape to a flared-out shape.

Example 113. The frame of example 112, wherein the frame is implanted within vasculature of an animal and wherein the liquid flow and pressure is systolic blood flow and pressure.

Example 114. A valvular prosthetic for improving stagnated blood flow, comprising: a plurality of interconnected struts that form a tubular frame, the tubular frame having an inflow side and outflow side, an inner luminal wall attached to the tubular frame; a set of leaflets within the tubular frame, the set of leaflets is attached to the tubular frame or attached to the inner luminal wall and separate the inflow side from the outflow side of the tubular frame; and a set of one or more inflatable bags attached to the inner luminal wall on the outflow side of the frame.

Example 115. The valvular prosthetic of example 114, wherein each bag of the set of one or more inflatable bags is composed of a biocompatible flexible material.

Example 116. The valvular prosthetic of example 114 or 115, wherein each bag of the set of one or more inflatable bags is filled with a compressed fluid component that changes volume based on pressure.

Example 117. The valvular prosthetic of example 116, wherein the set of one or more inflatable of bags are configured such that when the valvular prosthetic experiences a liquid flow and pressure through the inner luminal wall, each bag of the set of one or more inflatable bags is capable of expanding into an inflated state.

Example 118. The valvular prosthetic of example 117, wherein the valvular prosthetic is implanted within vasculature of an animal and wherein the liquid flow and pressure is systolic blood flow and pressure.

Example 119. A valvular prosthetic for improving stagnated blood flow, comprising: a plurality of interconnected struts that form a tubular frame, the tubular frame having an inflow side and outflow side; an inner luminal wall attached to the tubular frame; a set of leaflets within the tubular frame, the set of leaflets attached to the tubular frame or attached to the inner luminal wall and separate the inflow side from the outflow side of the tubular frame; and a set of one or more free-flowing sheets having attached to the inner luminal wall on the outflow side of the frame, each free-flowing sheet of the set of one or more free-flowing sheets having at least one free edge.

Example 120. The valvular prosthetic of example 119, wherein each free-flowing sheet of the set of one or more free-flowing sheets is composed of a biocompatible flexible material.

Example 121. The valvular prosthetic of example 120, wherein the set of one or more free flowing sheets are configured such that when the valvular prosthetic experiences a liquid flow and pressure through the inner luminal wall, each free-flowing sheet of the set of one or more free-flowing sheets is capable of moving with the liquid flow.

Example 122. The valvular prosthetic of example 121, wherein the valvular prosthetic is implanted within vasculature of an animal and wherein the liquid flow and pressure is systolic blood flow and pressure.

Example 123. A valvular prosthetic for improving stagnated blood flow, comprising: a plurality of interconnected struts that form a tubular frame, the tubular frame having an inflow side and outflow side; an inner luminal wall attached to the tubular frame; a set of leaflets within the tubular frame, the set of leaflets attached to the tubular frame or attached to the inner luminal wall and separate the inflow side from the outflow side of the tubular frame; and a plurality of flexible magnetically driven microprotrusions that are linearly aligned in a direction of flow, and attached to the inner luminal wall, wherein each flexible magnetically driven microprotrusion of the plurality of flexible magnetically driven microprotrusions has a positive magnetic pole and negative magnetic pole at a tip of the microprotrusion.

Example 124. The valvular prosthetic of example 123, wherein the plurality of flexible magnetically driven microprotrusions comprises a larger driver microprotrusion.

Example 125. The valvular prosthetic of example 123 or 124, wherein each microprotrusion tip of the plurality of flexible magnetically driven microprotrusions has a particular pole alignment such that each the pole face of each tip is the same charge as the adjacent tip pole face.

Example 126. The valvular prosthetic of example 124 or 125, wherein the larger driver microprotrusion can stimulate flexing of the rest of the plurality of flexible magnetically driven microprotrusions.

Example 127. A frame system for use within a valvular prosthetic, the frame system comprising: a plurality of interconnected struts that form a tubular frame that is self-expanding, wherein the tubular frame comprises a shape-memory material for providing a radial force; and a plurality of anti-torsion elements, wherein each anti-torsion element is a protrusion that extends laterally from at least a subset of the plurality of interconnected struts.

Example 128. The frame system of example 127, wherein each anti-torsion element is attached to a strut that is in an area of the frame that has a low density of struts.

Example 129. The frame system of example 127 or 128, wherein each anti-torsion element is fabricated as part of the frame design.

Example 130. The frame system of example 127 or 128, wherein each anti-torsion element is attached and secured to a strut by a means of attachment.

Example 131. The frame system of any one of examples 127 to 130, wherein at least a subset of anti-torsion elements of the plurality abuts or nearly abuts an adjacent strut when the frame is crimped.

Example 132. The frame system of any one of examples 127 to 131, wherein each anti-torsion element laterally extends a length between about 1.1 to 5 times the lateral width of the strut.

Example 133. The frame system of any one of examples 127 to 132, wherein each anti-torsion element has a vertical length between about 2% and 20% of the length of the strut.

Example 134. The frame system of any one of examples 127 to 133, wherein at least a subset of the plurality of anti-torsion elements comprises a marker for visualization.

Example 135. The frame system of any one of examples 127 to 134 further comprising a catheter, wherein the catheter houses the tubular frame, wherein each anti-torsion element mitigates the ability of a strut from twisting or contorting during loading of the tubular frame into the catheter.

Example 136. The frame system of example 135, wherein the catheter comprises an inner face having a polygonal contour.

Example 137. The frame system of example 136, wherein the tubular frame comprises a number of columnar segments, and wherein the number of columnar segments is equal to the number of sides of the polygonal contour.

Example 138. The frame system of example 136 or 137, wherein at least a subset of the plurality of interconnecting struts abuts or nearly abuts a side of the polygonal contour.

Example 139. The frame system of any one of examples 127 to 138, wherein the tubular frame is sterilized and packaged.

Example 140. A method of releasing a frame via a transcatheter technique, the method comprising: delivering a catheter and a tubular frame that is self-expanding to a site where the tubular frame is to be installed, wherein the tubular frame is loaded within the catheter, wherein the tubular frame comprises: a shape-memory material for providing a radial force; a plurality of interconnected struts; and a plurality of anti-torsion elements, wherein each anti-torsion element is a protrusion that extends laterally from at least a subset of the plurality of interconnected struts; distally advancing the tubular frame out of the catheter, resulting in an expansion of the tubular frame; and proximally reloading the tubular frame into the catheter, resulting in a crimping of the tubular frame.

Example 141. The method of example 140, wherein each anti-torsion element is attached to a strut that is in an area of the frame that has a low density of struts.

Example 142. The method of example 140 or 141, wherein each anti-torsion element laterally extends a length between about 1.1× and 5× the lateral width of the strut.

Example 143. The method of example 140, 141, or 142, wherein each anti-torsion element has a vertical length between about 2% and 20% of the length of the strut.

Example 144. The method of any one of examples 140 to 143, wherein at least a subset of the plurality of anti-torsion elements comprises a marker for visualization, the method further comprising: visualizing distally advancing or the proximally reloading of the self-expanding tubular frame via the marker for visualization and a visualization technique.

Example 145. A frame system for use within a valvular prosthetic, the frame system comprising: a plurality of interconnected struts that form a tubular frame that is self-expanding, wherein the tubular frame comprises a shape-memory material for providing a radial force; and a catheter, wherein the catheter houses the tubular frame, wherein the catheter comprises an inner face having a polygonal contour.

Example 146. The frame system of example 145, wherein the tubular frame comprises a number of columnar segments, and wherein the number of columnar segments is equal to the number of sides of the polygonal contour.

Example 147. The frame system of example 145 or 146, wherein at least a subset of the plurality of interconnecting struts abuts or nearly abuts a side of the polygonal contour.

Example 148. The frame system of example 145, 146, or 147 further comprising a plurality of anti-torsion elements, wherein each anti-torsion element is a protrusion that extends laterally from at least a subset of the plurality of interconnected struts.

Example 149. The frame system of example 148, wherein each anti-torsion element is attached to a strut that is in an area of the frame that has a low density of struts.

Example 150. The frame system of example 148 or 149, wherein at least a subset of anti-torsion elements of the plurality abuts or nearly abuts an adjacent strut when the frame is crimped.

Example 151. The frame system of example 148, 149, or 150, wherein each anti-torsion element laterally extends a length between about 1.1× and 5× the lateral width of the strut.

Example 152. The frame system of any one of examples 148 to 151, wherein each anti-torsion element has a vertical length between about 2% and 20% of the length of the strut.

Example 153. The frame system of any one of examples 145 to 152, wherein the tubular frame is sterilized and packaged.

Example 154. A method of releasing a frame via a transcatheter technique, the method comprising: delivering a catheter and a tubular frame that is self-expanding to a site where the tubular frame is to be installed, wherein the tubular frame is loaded within the catheter, wherein the catheter comprises an inner face having a polygonal contour, wherein the tubular frame is comprises a shape-memory material for providing a radial force and a plurality of interconnected struts; distally advancing the tubular frame out of the catheter, resulting in expansion of the tubular frame; and proximally reloading the tubular frame into the catheter, resulting in crimping of the tubular frame.

Example 155. The method of example 154, wherein the frame comprises a number of columnar segments, and wherein the number of columnar segments is equal to the number of sides of the polygonal contour.

Example 156. The method of example 154 or 155, wherein at least a subset of the plurality of interconnecting struts abuts or nearly abuts a side of the polygonal contour.

Example 157. The method of example 154, 155 or 156, wherein the tubular frame further comprises a plurality of anti-torsion elements, wherein each anti-torsion element is a protrusion that extends laterally from at least a subset of the plurality of interconnected struts.

Example 158. A cover for the fabrication of a tubular frame having a plurality of circumferences along the proximal-distal axis of the frame, the cover comprising: a precut sheet comprising a plurality of columnar segments that form a flower shape, wherein each columnar segment has a first end, a second end, and two lateral edges, wherein each columnar segment is laterally connected to two adjacent segments at the first end, wherein each columnar segment is not laterally connected from the two adjacent segments at the second end and for a majority of each of the two lateral edges.

Example 159. The cover of example 158, wherein the lateral edges of each columnar segment contours to match the plurality of circumferences along the proximal-distal axis of the frame.

Example 160. The cover of example 158 or 159, wherein the number of columnar segments of the plurality of segments of the cover match a number of columnar segments of the frame.

Example 161. The cover of example 158, 159 or 160, wherein the first end is a proximal end and the second end is a distal end.

Example 162. The cover of example 158, 159 or 160, wherein the first end is a distal end and the second end is a proximal end.

Example 163. The cover of any one of examples 158 to 162, wherein the precut sheet is composed of fabric, tissue, or film.

Example 164. A system of frame and cover, comprising: a tubular frame that extends a long a proximal-distal axis, wherein the tubular frame comprises: a plurality of columnar segments, each segment extending along the proximal-distal axis; a plurality of circumference lengths along the proximal distal axis; and a proximal end and a distal end; a cover surrounding the tubular frame, wherein the cover is composed of a precut sheet in a flower shape, wherein the cover comprises a plurality of columnar segments, wherein each columnar segment has a first end, a second end, and two lateral edges, wherein the two lateral edges of each columnar segment is laterally connected to lateral edges of two adjacent segments at the first end by the precut flower shape, wherein the two lateral edges of each columnar segment is laterally connected to lateral edges of two adjacent segments at the second end by a means of attachment.

Example 165. The system of example 164, wherein the majority of each of the two lateral edges of each columnar segment is laterally connected to lateral edges of two adjacent segments by the means of attachment.

Example 166. The system of example 164 or 165, wherein the means of attachment comprises one of: stitching, staples, or an adhesive.

Example 167. The system of example 164, 165 or 166, wherein the first end of the cover surrounds the proximal end of the tubular frame and the second end of the cover surrounds the distal end of the tubular frame.

Example 168. The system of example 164, 165 or 166, wherein the first end of the cover surrounds the distal end of the tubular frame and the second end of the cover surrounds the proximal end of the tubular frame.

Example 169. The system of any one of examples 164 to 168, wherein the lateral edges of each columnar segment of the cover contours to match the plurality of circumferences along the proximal-distal axis of the frame.

Example 170. The system of any one of examples 164 to 169, wherein the number of columnar segments of the plurality of segments of the cover match the number of columnar segments of the frame.

Example 171. The system of any one of examples 164 to 170, wherein the precut sheet is composed of fabric, tissue, or film.

While the above description contains many specific implementations of the various systems, devices, and methods, these should not be construed as limitations on the scope of these systems, devices, and methods, but rather as an example thereof. Accordingly, the scope of the claims should be determined not by the embodiments illustrated, but by the appended examples, the described alternatives, and their equivalents.

What is claimed is:

1. A prosthetic heart valve for replacing a native mitral or tricuspid valve, the prosthetic heart valve, comprising:
a support frame having an inlet end portion and an outlet end portion, the support frame comprising a plurality of interconnected struts;
a valve portion positioned within a lumen of the support frame, wherein the valve portion comprises a plurality of leaflets comprising pericardial tissue, wherein the valve portion provides unidirectional flow of blood through the lumen for replacing a function of the native valve; and
one or more bioresorbable elements disposed on or within the support frame.

2. The prosthetic valve of claim 1, wherein the support frame comprises a self-expanding frame that provides a radial force for anchoring to surrounding tissue, wherein resorption of the bioresorbable elements reduces the radial force.

3. The prosthetic valve of claim 1, wherein the one or more bioresorbable elements comprises bioresorbable elements that are located at an interconnection point of two or more struts.

4. The prosthetic valve of claim 1, wherein the support frame further comprises a plurality of appendages extending away from the support frame and wherein the one or more bioresorbable elements are located within one or more appendages of the plurality of appendages.

5. The prosthetic valve of claim 1, comprising an inner skirt attached to the support frame.

6. The prosthetic valve of claim 5, wherein the inner skirt is bioresorbable.

7. The prosthetic valve of claim 5, wherein the plurality of leaflets is attached to the inner skirt.

8. The prosthetic valve of claim 1 further comprising an outer skirt attached to the support frame.

9. The prosthetic valve of claim 8, wherein the outer skirt is bioresorbable.

10. The prosthetic valve of claim 1 further comprising a bioresorbable band that encircles the support frame.

11. The prosthetic valve of claim 10, wherein the bioresorbable band is coated with a reendothelialization-inducing biologic.

12. The prosthetic valve of claim 1 further comprising an anchoring system attached to the support frame.

13. The prosthetic valve of claim 12, wherein the anchoring system comprises a bioresorbable portion.

14. The prosthetic valve of claim 12, wherein the anchoring system comprises a plurality of anchoring arms with a curved portion capable of extending between chordae tendineae.

15. The prosthetic valve of claim 14, wherein at least one anchoring arm is bioresorbable.

16. The prosthetic valve of claim 12 further comprising a set of bioresorbable barbs attached to the support frame or attached to the anchoring system.

17. The prosthetic valve of claim 1 further comprising a bioresorbable constricting band that encircles the support frame.

18. The prosthetic valve of claim 1, wherein the support frame comprises a set of columnar segments and a set of bioresorbable connectors and wherein the set of bioresorbable connectors connect the set of columnar segments to form the support frame and wherein each columnar segment is in connection with two adjacent columnar sections via one or more bioresorbable connectors of the set.

19. The prosthetic valve of claim 1, wherein the support frame is capable of being compressed for placement within a sheath of a transcatheter delivery system.

20. The prosthetic valve of claim 1, wherein the support frame is sterilized and packaged.
